(19) United States
(12) Patent Application Publication
(10) Pub. No.: US 2026/0272989 A1
(43) Pub. Date: Sep. 17, 2026

(54) EXTRACELLULAR VESICLE MICRORNAS AND USES THEREOF

(71) Applicants: THE UNIVERSITY OF VERMONT AND STATE AGRICULTURAL COLLEGE, BURLINGTON, VT (US); UNITY HEALTH TORONTO, TORONTO (CA)

(72) Inventors: CLAUDIA CHIMISSO DOS SANTOS, TORONTO (CA); SARA ROLANDSSON ENES, LUND (SE); DANIEL J. WEISS, ESSEX JUNCTION, VT (US)

(21) Appl. No.: 18/871,960

(22) PCT Filed: Jun. 6, 2023
(86) PCT No.: PCT/US2023/067978

§ 371 Date: Dec. 5, 2024

Related U.S. Application Data
(60) Provisional application No. 63/349,551, filed on Jun. 6, 2022.

(51) Int. Cl. A61K 35/28; A61K 9/50; A61K 31/7088; C12N 5/0775; C12Q 1/6876

(52) U.S. Cl. CPC A61K 35/28; A61K 9/5068; A61K 31/7088; C12N 5/0662; C12Q 1/6876; C12Q 2600/158; C12Q 2600/178

(57) ABSTRACT

Disclosed herein are compositions comprising mesenchymal stromal cell-derived extracellular vesicle-associated miRNAs (MSC-EV-miRNAs) and their use in subjects diagnosed with acute respiratory distress syndrome (ARDS) and other inflammatory diseases. Also disclosed herein are methods for creating a differential MSC-EV-miRNA profile for any disease of interest (e.g., ARDS) and the use of miRNA profiles to create miRNA-based therapies. Additional aspects of the disclosure relate to primer mixes for the rapid detection of specific miRNA profiles, and kits thereof.

### RELATED APPLICATION

This application claims the benefit under 35 U.S.C. § 119(e) of U.S. provisional application No. 63/349,551, filed Jun. 6, 2022, which is incorporated by reference herein in its entirety.

### BACKGROUND OF THE INVENTION

Cell-based therapies utilizing mesenchymal stromal cells (MSCs) are being increasingly investigated as potential therapeutics for various diseases, including, for example, ischemic stroke, myocardial infarction, and acute respiratory distress syndrome (ARDS), including ARDS resulting from SARS-COV-2 infection, etc. However, despite significant pre-clinical success, results from recent human clinical studies remain variable, suggesting that new methods are needed to improve the therapeutic efficacy of MSC-based therapies (e.g., MSC-EVs).

### SUMMARY OF THE INVENTION

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways. The details of one or more embodiments of the invention are set forth in the accompanying Detailed Description, Examples, and Claims. Other features, objects, and advantages of the invention will be apparent from the description and from the claims.

In some aspects, the disclosure relates to a method for producing an EV-miRNA profile, comprising adding an activating agent to a culture of mesenchymal stromal cells (MSCs), determining whether at least one MSC extracellular vesicle-associated miRNA (MSC-EV-miRNA) is present in the culture of MSCs using a miRNA sequencing platform, and creating an EV-miRNA profile for the MSCs exposed to the activating agent based on the presence or absence of each miRNA, relative to a control. In some embodiments, the method further comprising using the MSC-EV-miRNA profile to identify a subject to receive the MSCs for the treatment of a disease. In some embodiments, the miRNA comprises hsa-miR-7107-5p. In some embodiments, the miRNA comprises hsa-miR-6803-5p. In some embodiments, the miRNA comprises hsa-miR-6798-5p. In some embodiments, the miRNA comprises hsa-miR-760. In some embodiments, the miRNA comprises hsa-miR-6727-5p. In some embodiments, the miRNA comprises hsa-miR-4763-3p. In some embodiments, the miRNA comprises hsa-miR-3652. In some embodiments, the miRNA comprises hsa-miR-885-3p. In some embodiments, the miRNA comprises hsa-miR-766-3p. In some embodiments, the miRNA comprises hsa-miR-3175. In some embodiments, the miRNA comprises hsa-miR-6893-5p. In some embodiments, the miRNA comprises hsa-miR-6875-5. In some embodiments, the miRNA comprises hsa-miR-6799-5p. In some embodiments, the miRNA comprises hsa-miR-6787-5p.

Some aspects of the disclosure relate to a method, comprising obtaining a first biological sample from a healthy first subject and a second biological sample from a second subject suspected of having a disease, culturing the first biological sample in a first culture of MSCs and a second biological sample in a second culture of MSCs, detecting whether at least one MSC-EV-miRNA is present in the first and/or second culture of MSCs using a next generation sequencing platform, using the next generation sequencing platform to create a differential EV-miRNA profile, and determining, based on the differential EV-miRNA profile, if the subject suspected of having the disease is a candidate for a MSC-based therapy.

Additional aspects of the disclosure relate to a pharmaceutical composition, comprising at least one mesenchymal stromal cell-derived extracellular vesicle associated miRNA (MSC-EV-miRNA), and a pharmaceutically acceptable excipient. In some cases, the pharmaceutical composition further comprises a lipid nanoparticle (LNP) encapsulating the at least one MSC-EV-miRNA. In certain embodiments, the pharmaceutical composition comprises LNPs encapsulating at least four MSC-EV-miRNAs. Yet, in some additional embodiments, still, the pharmaceutical composition comprises LNPs encapsulating at least 14 MSC-EV-miRNAs. In some cases, the LNP may comprise a targeting moiety. In some embodiments, the miRNA comprises hsa-miR-7107-5p. In some embodiments, the miRNA comprises hsa-miR-6803-5p. In some embodiments, the miRNA comprises hsa-miR-6798-5p. In some embodiments, the miRNA comprises hsa-miR-760. In some embodiments, the miRNA comprises hsa-miR-6727-5p. In some embodiments, the miRNA comprises hsa-miR-4763-3p. In some embodiments, the miRNA comprises hsa-miR-3652. In some embodiments, the miRNA comprises hsa-miR-885-3p. In some embodiments, the miRNA comprises hsa-miR-766-3p. In some embodiments, the miRNA comprises hsa-miR-3175. In some embodiments, the miRNA comprises hsa-miR-6893-5p. In some embodiments, the miRNA comprises hsa-miR-6875-5. In some embodiments, the miRNA comprises hsa-miR-6799-5p. In some embodiments, the miRNA comprises hsa-miR-6787-5p.

Some aspects of the disclosure relate to a cell therapy comprising a culture of engineered mesenchymal stromal cells configured to release a plurality of extracellular vesicles (MSC-EVs), wherein the plurality of MSC-EVs comprises hsa-miR-7107-5p, hsa-miR-6803-5p, hsa-miR-6798-5p, hsa-miR-760, hsa-miR-6727-5p, hsa-miR-4763-3p, hsa-miR-3652, hsa-miR-885-3p, hsa-miR-766-3p, hsa-miR-3175, hsa-miR-6893-5p, hsa-miR-6875-5p, hsa-miR-6799-5p, hsa-miR-6787-5p. In some embodiments, the cell therapy comprises MSC-EVs comprising hsa-miR-760, hsa-miR-3175, hsa-miR-885-3p, and hsa-miR-766-3p.

Certain aspects of the disclosure further relate to a method of producing therapeutic mesenchymal stromal cells (MSC), the method comprising obtaining a first biological sample from a healthy first subject and a second biological sample from a second subject suspected of having a disease, culturing the first biological sample in a first culture of MSCs and a second biological sample in a second culture of MSCs, isolating hMSC-associated extracellular vesicles (hMSC-EVs) from the cell culture, determining whether at least one EV-associated miRNA (EV-miRNA) is present in the hMSC-EVs using a next generation sequencing platform, using the next generation sequencing platform to create a differential EV-miRNA profile, and engineering the therapeutic MSC to overexpress one or more miRNAs identified in the EV-miRNA profile. In some embodiments, the method further comprises delivering the therapeutic MSC to the subject. In some cases, the method may comprise therapeutic MSCs that overexpress hsa-miR-760, hsa-miR-3175, hsa-miR-885-3p, and hsa-miR-766-3p.

Aspects of the current disclosure are also related to an oligonucleotide primer mix comprising one or more oligonucleotides, wherein each oligonucleotide is configured to hybridize to a single miRNA of interest, and wherein the miRNA of interest is selected from the group consisting of hsa-miR-7107-5p, hsa-miR-6803-5p, hsa-miR-6798-5p, hsa-miR-760, hsa-miR-6727-5p, hsa-miR-4763-3p, hsa-miR-3652, hsa-miR-885-3p, hsa-miR-766-3p, hsa-miR-3175, hsa-miR-6893-5p, hsa-miR-6875-5p, hsa-miR-6799-5p, hsa-miR-6787-5p.

Some aspects of the current disclosure also relate to a kit for detecting the presence of at least one mesenchymal stromal cell derived extracellular vesicle-associated miRNAs (MSC-EV-miRNA) in a biological sample, comprising a primer mix comprising one or more oligonucleotides, wherein each oligonucleotide is configured to hybridize to a single miRNA of interest, and wherein the miRNA of interest is selected from the group consisting of hsa-miR-7107-5p, hsa-miR-6803-5p, hsa-miR-6798-5p, hsa-miR-760, hsa-miR-6727-5p, hsa-miR-4763-3p, hsa-miR-3652, hsa-miR-885-3p, hsa-miR-766-3p, hsa-miR-3175, hsa-miR-6893-5p, hsa-miR-6875-5p, hsa-miR-6799-5p, hsa-miR-6787-5p, a reagent for performing a nucleic acid assay to detect the at least one MSC-EV-associated miRNAs using the nucleic acid pair, and instructions for performing the assay to detect the at least one MSC-EV-associated miRNAs. In some embodiments, the includes at least four oligonucleotide for detecting at least four of the MSC-EV-associated miRNAs. In certain additional embodiments, the kit includes at least eight oligonucleotides for detecting at least four of the MSC-EV-associated miRNAs.

### BRIEF DESCRIPTION OF THE DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1 is the of the experimental design schematic. Human mesenchymal stromal cells (hMSCs) were exposed in vitro to bronchoalveolar lavage fluid (BALF) from healthy volunteers (HVs) or acute respiratory distress syndrome (ARDS) patients for 5 hours. Extracellular vesicles (EVs) contained in supernatant were isolated, and characterized for particle number, size distribution, and expression of characteristic cell surface markers. MiRNAs from purified EVs were sequenced and analyzed. MiRNAs that met statistical cutoffs for differential expression were further analyzed to identify their predicted gene targets and putative functional enrichment. In parallel, Partial Least Square Discriminant Analysis (sPLS-DA) was used to identify miRNAs that classify samples into specific treatment groups. Overlap Analysis was used to overlay results from DEA and sPLS-DA to identify 14 differentially expressed miRNAs able to classify samples into treatment response groups. Network analysis identified 4 miRNAs as putative “hub” regulators of involved in top biological pathways. EVs contained in BALF samples from ARDS patients and HVs were isolated and characterized for particle number, size distribution, and expression of tetraspanin markers. MiRNAs from purified BALF-derived EVs were sequenced and analyzed for differential expression.

FIGS. 2A-2D show EVs from BALF stimulated hMSCs differ in size distribution and tetraspanin expression compared to EVs isolated directly from BALF. FIG. 2A shows EVs secreted by hMSCs exposed to serum-free medium alone (control) or to BALF collected from HVs or patients with ARDS are similar in size (50-200 μm) and number (n=3, 4, and 4, for control, HV and ARDS, respectively). Data for each condition are means±SEM per 100 μm bin, as detailed in the methods. AUC for each condition is not statistically significant, p>0.05. FIG. 2B shows image flow cytometry of the same EV samples characterized in FIG. 2A, and shows both HV and ARDS BALF exposure decreased CD63 positive EVs overall compared to control, with HV BALF exposure resulting in lower CD63 expression than ARDS BALF exposure. No significant difference in CD81 expression between groups and no CD9 expression was detected in any group. * different than control and different than HV. FIG. 2C shows EVs present in BALF alone versus control (serum-free medium) show a different pattern than EVs obtained from BALF-stimulated hMSCs: EVs of larger size are more prevalent and ARDS BALF has a higher number of EVs (n=2, 3, 3 for control, HV and ARDS, respectively). Data for each condition are means±SEM per 100 μm bin. The AUC for ARDS is significantly higher than HV and control as indicated by asterisks, p<0.05. FIG. 2D shows image flow cytometry of the same EV samples characterized in FIG. 2B, and shows the presence of CD9 in addition to CD63 and CD81, but no differences in expression of these tetraspanins between groups. Abbreviations: ARDS, acute respiratory distress syndrome; AUC, Area Under the Curve; EVs, extracellular vesicles; HV, healthy volunteer; NTA, Nanotracking analysis.

FIGS. 3A-3E show EVs derived from resting control hMSCs contain miRNA involved in cell cycle regulation. FIG. 3A is a Significance Analysis of Microarray (SAM) output plot showing miRNAs that were over-represented in EVs derived from control hMSCs. The δ-value in SAM was set to 7 (best delta selected by software for lowest False Discovery Rate [FDR]), and the FDR was 0(%). A total of 770 miRNAs were observed to be over-represented above the expected rate compared to the average expression of all miRNAs across replicates (above the upper delta line). MiRNAs between the upper and lower delta line were not over-represented. A total of 48 miRNAs were markedly over-represented (score ≥20 above cut-off line, in square dashed line). Median Number of False Positives=0; Tail strength (%)=99.4; se (%)=153.7. FIG. 3B shows the results of the plot of functional enrichment analysis for the 48 miRNAs that scored >20 (expected vs observed) performed in miRNet. Putative relationships were obtained using DIANA-TarBase v8 (collection of experimentally supported miRNA-gene interactions). Enriched terms (filtered by hypergeometric test cut-off=adjusted p-value≤0.05). FIG. 3C shows the top 10 REACTOME terms. The Log 10 q-value (FDR) of the enrichment score is plotted. Top network for miRNAs and putative targets involved in cell cycle were generated using “shortest path finder”, filter based on degrees (cut-off=3) and minimal network algorithm. FIG. 3D shows miRNAs and FIG. 3E shows their known targets. Square nodes are miRNAs (D, N=24) and circles are genes (N=61). Cell cycle related targets are highlighted (circles, N=24). Edges represent empirically demonstrated direct interaction between nodes (TarBasev8.0). Size of node reflects number of direct contacts. MiRNAs are labelled by their miRNA base ID and genes using their official HUGO symbol.

FIGS. 4A-4C show MiRNAs are differentially expressed in EVs derived from hMSCs exposed to HV or ARDS BALF. Volcano plots of differentially expressed miRNAs (fold change (FC) in normalize counts >2 and false discovery rate (FDR) <0.05) for pairwise comparisons: FIG. 4A shows control versus healthy volunteer (HV); FIG. 4B shows ARDS versus control, and FIG. 4C shows ARDS versus HV. Total number of miRNAs fulfilling differentially expression criteria are stated for each plot. Individual differentially expressed miRNAs are plotted as circles and colored by FC. In the volcano plots, non-differentially expressed miRNAs are shown as gray dots and dashed lines correspond to FC and FDR cut-offs. TarBase v8 predicted concordant regulated pathways for over and under expressed miRNAs are shown as horizontal bar graphs and colored by expected direction of change. The top 10 enriched terms ranked by adjusted p-value <0.05 (expressed as −Log 10 of q-value) from hypergeometric test are presented.

FIGS. 5A-5G show different miRNA expression patterns are observed between control, HV, and ARDS treatment groups. FIG. 5A is a Venn diagram showing overlap in patterns of miRNA expression across all three pairwise comparisons. FIGS. 5B-5D show the overlap of differential expression patterns identified in miRNAs over-represented in all comparisons against control-derived EVs in FIG. 5B, those over-represented in comparisons against HV in FIG. 5C, and those differentially expressed after exposure to BALF from ARDS patients in FIG. 5D. FIGS. 5E-5G are cordant regulated pathways as predicted by MiRNet and are shown as horizontal bar graphs of the top 10 enriched terms as filtered by hypergeometric test and adjusted p-value <0.05 (expressed as −Log 10 of q-value). ARDS, acute respiratory distress syndrome; C, serum-free medium control; FC, fold change; HV, healthy volunteer.

FIGS. 6A-6F show 52 MiRNAs are commonly found in EVs derived from hMSCs exposed to the different experimental conditions. FIG. 6A-6B are Venn diagrams showing the overlap of 13 and 52 miRNAs that are differentially expressed across all treatment groups-even when considering the combined effect of control plus HV (ARDS vs [Control+HV]). FIG. 6C (N=13) and FIG. 6D (N=52; Table 2) show the top 10 pathways from the overlap miRNAs that are predicted to be impacted by miRNA regulation ranked by hypergeometric test cut-off=adjusted p-value≤0.05 (miRNet Tarbase v8). The Log 10 q-value (FDR) of the enrichment score is plotted. In FIGS. 6E-6F, the Log 2 fold change (FC) for miRNAs that passed statistical cut-off (FC≥2.0; FDR≤0.05) is plotted for each miRNA for each comparison beside the heat map to underscore consistency in the pattern of expression for these miRNAs in EVs derived from hMSCs. Individual miRNAs are shown. Color legend is Log 2 FC. ARDS, acute respiratory distress syndrome; C; control; Control, serum-free medium; EVs, extracellular vesicles; hMSCs, human mesenchymal stromal cells; HV, healthy volunteer; miR, microRNA.

FIGS. 7A-7C show the top EV-miRNAs over-represented in hMSC-derived EVs are involved in inflammatory and injury cell pathways. FIG. 7A is a Venn diagram showing 52 miRNAs, present in EVs derived from control hMSCs that were also differentially expressed across all comparisons: ARDS vs control, ARDS vs HV, and ARDS vs HV+control. Functional enrichment analysis demonstrated that these 52 miRNAs are involved in various pathways important in the regulation of inflammation and injury including but not limited to regulation of the inflammasome as shown in FIG. 7B, and cell stress pathways as shown in FIG. 7C. Network analysis for these two pathways in FIGS. 7B-7C show miRNAs associated with the network and their putative target genes ARDS, acute respiratory distress syndrome; control, serum-free medium; EVs, extracellular vesicles; hMSC, human mesenchymal stromal cells; HV, healthy volunteer.

FIG. 8A shows the top 20 (from 52) hMSC-derived EV miRNAs demonstrate different expression patterns following HV or ARDS BALF exposure. Box plots showing change in normalized miRNA counts (Log 2) in EVs derived from control hMSCs (red), treated with BALF from HVs and ARDS patients. Line in box is the median quartile, squares are upper and lower quartiles and whiskers are maximum and minimum range. ARDS, Acute Respiratory Distress Syndrome; Control, serum-free medium; EVs, extracellular vesicles; hMSCs, human mesenchymal stromal cells; HV, healthy volunteer; miR, microRNA.

FIGS. 8B-8C show differentially expressed miRNAs in EVs derived from BALF-exposed hMSC-derived EVs vs EVs isolated directly from BALF. Box plots showing representative changes in normalized counts (Log 2) of miRNAs deemed to be differentially expressed in BALF vs those found to be differentially expressed in EVs derived from control hMSCs, treated with BALF from HV, and ARDS patients. Line in box is the median quartile, squares are upper and lower quartiles and whiskers are maximum and minimum range. BALF, bronchoalveolar lavage fluid samples; control, serum-free medium; HV, healthy volunteer; ARDS, acute respiratory distress syndrome; miR, microRNA; hMSC, human mesenchymal stromal cells; EVs, extracellular vesicles.

FIGS. 9A-9C show supervised discriminate analysis identified miRNAs able to classify samples into experimental treatment groups. FIG. 9A shows a sparse PLS-DA plot where each sample is a point colored by class and lines extend from the group centroid to the individual samples. 20 miRNAs were selected for maximal discrimination between classes. FIG. 9B shows an ROC curve with AUC for all-vs-one comparisons averaged over all cross-validations and based on predicted maximum distances. FIG. 9C are box plots showing normalized counts of all 20 miRNAs selected as classifiers by sPLS-DA. Median quartile are shown as well as upper and lower quartiles; whiskers are maximum and minimum range. ARDS, acute respiratory distress syndrome; Control, serum-free medium; HV, healthy volunteer; miR, microRNA.

FIGS. 10A-10C show 14 miRNAs overlap between discriminant and differential analyses. FIG. 10A is a Venn diagram showing overlap between discriminant analyses. FIGS. 10B-10C show interaction networks for the cellular response to stress or to Wnt signaling. MiRNA 766-3p, miRNA-760, miR885-3p, and miRNA-3175 are shown. Genes in the network but not linked to the cellular response to stress or in signaling by Wnt are shown as small circles. Edges indicate direct interactions between nodes (Tarbase v8).

### DETAILED DESCRIPTION OF THE INVENTION

Cell-based therapies utilizing mesenchymal stromal cells (MSCs) are being increasingly investigated as potential therapeutics for various diseases, including, for example, ischemic stroke, myocardial infarction, and acute respiratory distress syndrome (ARDS), including ARDS resulting from SARS-COV-2 infection. These approaches are based on the paracrine anti-inflammatory actions of MSCs, which in the case of ARDS, may reduce intra-alveolar inflammation, facilitate endothelial repair, increase alveolar fluid clearance, and regulate lung epithelial and endothelial permeability.

One mechanism by which MSCs may regulate paracrine signaling, e.g., during a disease-mediated inflammatory response, is to release extracellular vesicles (EVs), including exosomes and microvesicles. EVs are particles that are delimited by a lipid bilayer, cannot replicate, and encapsulate a cargo comprising a plurality of signaling molecules (i.e., proteins, lipids, and nucleic acids such as mRNA, micro-RNAs or miRNAs, long non-coding RNAs, DNA, and various other metabolites). Without wishing to be bound by theory, it is generally believed that MSC-EVs exert their functions through the transfer of the cargo, which act as paracrine signaling agents to communicate between adjacent and/or distant cells.

Several pre-clinical studies have consistently demonstrated that both systemic or intratracheal MSC administration, and more recently systemic administration of MSC-EVs, results in improved clinical outcomes (e.g., amelioration of lung injury in models of ARDS). However, despite the success of MSC cell-based therapies to treat human disease in various pre-clinical models (e.g., mouse models of ARDS), results from recent human clinical studies remain variable, suggesting that new methods are needed to improve the therapeutic efficacy of MSC-based therapies (e.g., MSC-EVs).

As described herein, MSC-based paracrine responses, and thus potential therapeutic actions, may differ depending on the cellular microenvironment encountered by the MSCs. For instance, as shown in Example 1, human MSCs (hMSCs) cultured with bronchoalveolar lavage fluid (BALF) obtained from subjects diagnosed with ARDS (ARDS BALF) affected hMSC-EV release, expression of characteristic EV cell surface tetraspanin protein markers, and miRNA content differently compared to hMSC cultured with BALF obtained from healthy subjects (HV BALF). This discovery permits creation of differential EV-miRNA profiles (e.g., using target prediction, functional analyses, and visual exploration) that highlight alterations in miRNAs known to be relevant to hMSC therapeutic effects (e.g., paracrine effects), including those involved in the regulation of inflammation, cell-cycle, proliferation, and apoptosis.

As such, in some aspects, the current disclosure solves the aforementioned problems by providing a method for determining disease specific MSC-EV-miRNA profiles. The method, in some embodiments, may be used to identify miRNAs that serve as regulators of MSC paracrine responses. For example, in some embodiments, the method may be used to identify miRNAs that regulate MSC paracrine responses in ARDS.

In other aspects, the current disclosure solves the aforementioned problems by providing a disease-specific MSC-EV-miRNA profile that may be used, for example, to identify diseases most likely to benefit from MSC-based cell therapies. For instance, in some embodiments, the method comprises obtaining biological samples from subjects with and without a disease, adding them to cultures of MSCs, and profiling the miRNA expression patters and creating a creating a differential MSC-EV-miRNA profile. For example, disease-specific differential MSC-EV-miRNA profiles generated from exposing MSCs cultures to ARDS BALF or HV BALF comprise a plurality of miRNAs involved in Wnt signaling and other cellular responses to stress, suggesting that MSC-based cell therapies may have therapeutic benefits in the setting of ARDS. On the other hand, a MSC-EV-miRNA profile (generated for a specific disease of interest) that displays limited changes in miRNA expression patterns may suggest that MSC-based cell therapies may not have therapeutic benefits in the specific disease of interest.

The current disclosure, in some aspects, further solves the aforementioned problems by providing methods to tailor the contents of the MSC-EVs, for example, using genetic engineering. In some embodiments, the method comprises creating a differential MSC-EV-miRNA profile and identifying miRNAs that counteract the pathophysiology of various diseases, such as, for example, the pro-inflammatory response associated with ARDS. In some cases, the MSCs may be genetically modified, for example, to overexpress and package the identified miRNAs into EVs, thus improving the therapeutic efficacy of MSCs following administration to a subject in need thereof.

In yet another aspect, the current disclosure solves the aforementioned problems by identifying factors in the EVs (e.g., miRNAs) that may be used as acellular therapeutics. This may be particularly useful, for example, in situations where the EV-miRNA profile identifies a subset of miRNAs that regulate multiple paracrine responses. The subset of miRNAs, may in some cases, be encapsulated within a microcarrier, such as a lipid nanoparticle, optionally comprising a targeting moiety, and administered via intravenous, intraosseous, intramuscular, intraperitoneal, or via subcutaneous injection. In some cases, the EV may be used as the drug delivery vehicle.

### Methods of Generating MSC-EV-miRNA Profiles

Aspects of the current disclosure relate to a method of producing a differential MSC-EV-miRNA profile. In some embodiments, the method comprises adding an activating agent to a culture of mesenchymal stromal cells (MSCs). In some cases, the activating agent may comprise various disease-associated stimuli, e.g., chemical, mechanical, temperature, light, etc. For example, in some embodiments, the activating agent comprises a biological sample (i.e., a chemical stimuli) obtained from a subject with a disease; in other cases, the biological sample may be obtained from a healthy subject. Exemplary embodiments of biological samples include, but are not limited to, blood, serum, urine, semen, synovial fluid, interstitial fluid (i.e., lymphatic fluid), bile, pus, phlegm, saliva, rheum, cerebrospinal fluid, blood plasma, transudate, tears, gastric juices, amniotic fluid, aqueous humor, breast milk, cerumen, chyle, exudates, mucus, pericardial fluid, peritoneal fluid, pleural fluid, sebum, serous fluid, sputum, sweat, vomit, bronchoalveolar lavage fluid (BALF), etc.

The biological sample, according to other embodiments, may comprise all human tissues (e.g., fresh, frozen, fixed, or processed) and/or all human blood (e.g., peripheral and/or umbilical cord blood) and blood byproducts (e.g., serum, plasma, buffy coat) and/or all human biofluids (e.g., sputum, urine, bile) and/or human primary cells derived from human biosamples, and/or any DNA derived from individual donors.

In other embodiments, an activating agent may comprise a solution comprising one or more agents known by those of skill in the art to be associated with a disease (e.g., inflammation). In certain embodiments, the activating agent comprises one or more immune and/or inflammatory cells, such as Th1 cells, CD4+ cells, macrophages, dendritic cells, or any combination thereof. In other cases, the activating agent comprises one or more types of cytokines, such as, for example, a lymphokine (cytokines made by lymphocytes), a monokine (cytokines made by monocytes), a chemokine (cytokines with chemotactic activities) and/or an interleukin (cytokines made by one leukocyte but act on other leukocytes). Non-limiting examples include, but are limited to, interleukin-1 (IL-1), interleukin-2 (IL-2), interleukin-12 (IL-12), interleukin-17 (IL-17), interleukin-18 (IL-18), IFN-gamma, and TNF-alpha.

In some embodiments, an activating agent comprises a mechanical stimuli. For example, many solid tumors (e.g., glioblastomas, prostate, breast) exhibit high solid stresses that are driven by a hypoxic tumor microenvironment. In such cases, the activating agent may comprise an oxygen tension of, for example, a culture of MSCs. For example, in some cases, the method comprises lowering the oxygen tension of the culture of MSCs from ~20% oxygen to between 1% to 5% oxygen (percents are volume percents). In some embodiments, the method comprises lowering the oxygen tension to greater than or equal to 1%, greater than or equal to 2%, greater than or equal to 3%, greater than or equal to 4%, greater than or equal to 5%, etc., of the total gas volume. In other embodiments, the method comprises lowering the oxygen tension to less than or equal to 5%, less than or equal to 4%, less than or equal to 3%, less than or equal to 2%, less than or equal to 1%, etc., of the total gas volume.

Other mechanical stresses, e.g., shear stress or cyclic mechanical strain, may also be used as an activating agent. For instance, in some embodiments, MSCs may be grown on microcarriers and expanded in a stir-batch bioreactor, wherein the act of stirring induces a fluid shear stress on the cultured MSCs. Other types of mechanical stimulation may include acoustic activation (e.g., subjecting a culture of MSCs to ultrasonication) and/or repeated exposure to stretch or compressive forces (e.g., after growing MSCs on a flexible substrate). Exposure to various temperature cycles and wavelengths of light may also be used to augment the MSC microenvironment and stimulate changes in EV miRNA content. Combinations are also possible, e.g., a culture of MSCs may be exposed to an activator solution and low oxygen tension.

After exposing a culture of MSC with an activating agent, mesenchymal stromal cell extracellular vesicles (MSC-EVs) may be harvested and the miRNA content within said MSC-EVs determined. MSC-EVs may be harvested from the cell culture media using commercially available kits, such as, Exosome Isolation Kits (Miltenyli Biotec) and exoEasy Maxi Kit (Qiagen) or via any other technique known by those of skill in the art, such as, for example, magnetic isolation, ultracentrifugation, differential ultracentrifugation, sequential centrifugation, size-based fractionation (e.g., tangential flow filtration and size exclusion chromatography). Following isolation of the EVs, the relevant miRNAs may be isolated (e.g., using standard laboratory practices). In most cases, commercially available kits may be purchased and used to extract the miRNAs (e.g., exoRNeasy Midi and Maxi kits from Qiagen). miRNAs may then be sequenced using a nucleic acid-based detection assay (e.g., using Pyrosequencing on the 454 Life Sciences platform, polymerase-based sequence-by synthesis on the Illumina platform, the sequencing by ligation on the ABI Solid Sequencing platform, or the HTG EdgeSeq miRNA Whole Transcriptome Assay) to identify miRNAs present in the EV. The latter may include identifying known miRNAs and identification of novel miRNAs (e.g., by performing miRNA alignment analyses).

In some embodiments, a miRNA sequencing analysis measures the expression of between 1000 and 5000 human miRNA transcripts. In some embodiments, the sequencing analysis measures greater than or equal to 1000, greater than or equal to 2000, greater than or equal to 3000, greater than or equal to 4000, greater than or equal to 5000, etc., human miRNA transcripts. In other embodiments, the sequencing analysis measures less than or equal to 5000, less than or equal to 4000, less than or equal to 3000, less than or equal to 2000, or less than or equal to 1000 human miRNA transcripts.

Following miRNA sequencing of MSC-EV-miRNAs, a miRNA differential expression analysis may be performed. Differential expression analysis is useful, for example, for comparing the effects of two different activating agents on the MSC-EV-miRNA content (e.g., ARDS BALF vs HV BALF) or the effect of a single activator over various time points, etc. For instance, in some embodiments, treatment of a culture of MSC with ARDS BALF induces over-expression of hsa-miR-7107-5p, hsa-miR-6803-5p, hsa-miR-6798-5p, hsa-miR-760, hsa-miR-6727-5p, hsa-miR-4763-3p, hsa-miR-3652, hsa-miR-885-3p, hsa-miR-766-3p, hsa-miR-3175, hsa-miR-6893-5p, hsa-miR-6875-5p, hsa-miR-6799-5p, hsa-miR-6787-5p, among others, relative MSCs cultured with HV BALF, respectively.

Differential expression analysis (DEA) may be performed using any software known to those of skill in the art, e.g., DESeq2 in R (R package “DESeq2”). Without wishing to be bound by theory, in some cases, a variance stabilization transformation may be used to prepare miRNA sequence data for differential expression analyses. In some embodiments, the differential expression analysis comprises running a significance analyses of microarrays (SAM) of normalized read counts, using a one class analysis approach, to identify miRNAs over-represented in EVs derived from control samples. In other embodiments, the differential expression analysis comprises setting a delta value to 7 (i.e., to ensure the lowest False Discovery Rate [FDR]) using 1000 permutations. In some cases, miRNAs deemed to be differentially expressed (DE) exhibit a greater than or equal to 1.2-fold change in expression, greater than or equal to 1.5-fold change in expression, greater than or equal to 1.7-fold increase in expression, greater than or equal to 2-fold change in expression, greater than or equal to 2.5-fold change in expression, greater than or equal to 3-fold change in expression, etc. In other embodiments, the miRNAs deemed to be differentially expressed exhibit a less than or equal to 3-fold change in expression, less than or equal to 2.5-fold change in expression, less than or equal to 2-fold change in expression, less than or equal to 1.7-fold change in expression, less than or equal to 1.5-fold change in expression, less than or equal to 1.2-fold change in expression, etc. In some cases, miRNAs deemed to be differentially expressed (DE) may be chosen based on a greater than or equal to 2-fold change in expression (i.e., the mean expression across samples >50 read counts) and an adjusted P-value (or FDR) <0.05 after correction for multiple comparisons.

In some embodiments, the method further comprises preforming a target prediction and enrichment analysis. Target prediction analysis identifies the miRNAs target mRNA and helps to provide an understanding of the genes or networks of genes whose expression they regulate. Without wishing to be bound by theory, target prediction analysis may be performed using commercially available software (e.g., using software such as Reactome, RNA22, TargetScan, miRanda, PicTar, miRNet, etc.,) and generally involves (1) determining miRNA: mRNA binding pairs. This may be done by identifying complementarity between the miRNA sequences at the 3′-UTR of the mRNA sequence; (2) determining the degree of conservation of miRNA: mRNA binding pairs across species, and (3) observing for evidence of miRNA targeting in mRNA-seq or protein expression data, wherein if the miRNA expression is high, the gene and protein expression of its target gene should be low.

Following identification of the target mRNAs, a gene set enrichment analysis may be performed, according to some embodiments. As those of skill in the art may appreciate, such analyses may be used to, for example, identify classes of genes or proteins that are over-represented in a large set of genes or proteins, and may have an association with disease phenotypes. As with other ‘omics-platforms,’ there are a number of commercially available tools for performing gene enrichment analyses. Exemplary tools may include, but are not limited to, NASQAR, PlantRegMap, Blas2Go, GREAT, MSigDB, etc.).

Additional aspects of the method comprise performing a sparse partial least squares discriminant analysis (sPLS-DA). Without wishing to be bound by theory, sPLS-DA is a statistical method that uses a linear regression model to find the fundamental relationship between a response (e.g., y-variable, ARDS positive or negative patient) and independent variables (e.g., x-variables, miRNA expression). This is accomplished by using a latent variable approach, which allows the categorical response variable (e.g., ARDS positive or negative patient sample) to be analyzed as though it was a continuous variable. This allows the model to perform variable selection and classification in a one-step procedure. In other words, performing sPLS-DA on sequencing data (e.g., miRNA sequencing data) may be used to create a mathematical equation to correlate the x-variables (e.g., EV-miRNAs) and y variables (e.g., ARDS positive, ARDS negative, controls). This equation may then be used, for example, to classify a disease of interest as likely to be responsive or unresponsive to MSC-cell-based therapies. The equation may also be used to formulate an acellular pharmaceutical therapy, for example, by providing the identity of the therapeutic miRNAs and an estimate of their relative concentrations (e.g., for encapsulation within a lipid nanoparticle).

In some embodiments, performing a sPLS-DA analysis comprises obtaining a miRNA sequence library. In some embodiments, the miRNA sequence library may comprise between 1000 and 5000 human miRNA transcripts. In some embodiments, the library comprises greater than or equal to 1000, greater than or equal to 2000, greater than or equal to 3000, greater than or equal to 4000, greater than or equal to 5000, etc., human miRNA transcripts. In other embodiments, the library comprises less than or equal to 5000, less than or equal to 4000, less than or equal to 3000, less than or equal to 2000, or less than or equal to 1000 human miRNA transcripts.

In some embodiments, performing a sPLS-DA analysis comprises randomly splitting a miRNA sequencing data into a training set and a test set using a 0.7/0.3 split. In other embodiments, the ratio of the training set/test set may be greater than or equal to 0.1/0.9, greater than or equal to 0.2/0.8, greater than or equal to 0.3/0.7, greater than or equal to 0.4/0.6, greater than or equal to 0.5/0.5, greater than or equal to 0.6/0.4, greater than or equal to 0.7/0.3, greater than or equal to 0.8/0.2, greater than or equal to 0.9/0.1. In some embodiments, the ratio of miRNAs in the training set may be less than or equal to 0.9/0.1, less than or equal to 0.8/0.2, less than or equal to 0.7/0.3, less than or equal to 0.6/0.4, less than or equal to 0.5/0.5, less than or equal to 0.4/0.6, less than or equal to 0.3/0.7, less than or equal to 0.2/0.8, or less than or equal to 0.1/0.9.

The method, in some embodiments, comprises performing an overlap analysis and/or a network analysis. Without wishing to be bound by theory, overlap analyses, may be useful, for example, when comparing differentially expressed genes between various experimental groups and/or analysis techniques (e.g., differential expression analysis versus sPLS-DA). Network analysis may be used, for example, to visualize predicted ‘direct’ interactions between differentially expressed miRNAs of interest and their target genes. Such approaches may permit the identification of one or more sub-sets of miRNAs that, for example, serve as master regulators or ‘hubs’ of MSC paracrine signaling (e.g., for a given activating agent, such ARDS BALF).

### ARDS Specific MSC-EV-miRNA Profiles

Aspects of the current disclosure relate, in some cases, to using the aforementioned method to identify an MSC-EV-miRNA profile for a disease of interest, such as, for example, ARDS. For instance, Examples 1 through 8 highlight the use of the method to determine a differential MSC-EV-miRNA profile for subjects with acute respiratory distress syndrome (ARDS). Accordingly, in some cases, the MSC-EV-miRNA profile for ARDS includes, but is not limited to, the following 14 differentially regulated miRNAs: hsa-miR-7107-5p, hsa-miR-6803-5p, hsa-miR-6798-5p, hsa-miR-760, hsa-miR-6727-5p, hsa-miR-4763-3p, hsa-miR-3652, hsa-miR-885-3p, hsa-miR-766-3p, hsa-miR-3175, hsa-miR-6893-5p, hsa-miR-6875-5p, hsa-miR-6799-5p, hsa-miR-6787-5p, wherein these miRNAs collectively regulate processes such inflammation, cell-cycle, proliferation, apoptosis, and Wnt signaling. In some embodiments, the differentially regulated miRNAs may be further screened for a reduced set of miRNAs that work together to form a putative in-silico regulatory network that act as putative hub regulators for the entire set of gene targets (which in the case of the ARDS profile is ~1259 putative targets). In the case of the MSC-EV-miRNA profile (generated from ARDS BALF and HV BALF), the reduced set of miRNAs, in some embodiments, comprises miRNA-760, miRNA-3175, miRNA-885-3p, and miRNA-766-3p.

Other diseases of interest may also be profiled using the aforementioned method. For example, MSC-EV-miRNA profiles may be generated for other lung diseases (e.g., chronic pulmonary disease, chronic pulmonary obstructive disease, emphysema, asthma, etc.), neurological diseases (e.g., epilepsy, post-traumatic brain injury, brain damage in pre-term neonates, and stroke), ischemic diseases (e.g., myocardial infarction, chronic renal failure respiratory failure), arthritic diseases (e.g., osteoarthritis and rheumatoid arthritis), infectious conditions, various ophthalmic diseases, and cancers. In some embodiments, profiling MSC-EV contents during regenerative processes (e.g., regeneration of bone, liver, heart, muscles, blood cells) is also possible.

### Methods of Using MSC-EV-miRNA Profiles

Aspects of the current disclosure relate, in some cases, to a method to identify a disease likely to benefit from MSC therapy, such as for example, cancer immunotherapy. For instance, in some embodiments, the method comprises obtaining a first biological sample from a healthy first subject and a second biological sample from a subject suspected of having a disease (e.g., cancer, ARDS, myocardial infarction). In other embodiments, the method comprises adding the first biological sample and the second biological sample to a first culture of MSCs and a second culture of MSCs, respectively, and detecting whether at least one MSC-EV-miRNA is present in the first and/or second culture of MSCs using, for example, next generation sequencing (e.g., microRNA-Seq). As described elsewhere herein, a series of analyses may be done following miRNA sequencing (e.g., functional prediction, overlap analyses, and discriminant analysis) to create a disease specific differential EV-miRNA profile based on the presence or absence of each miRNA (relative to a control). Differential MSC-EV-miRNA profiles that indicate over-expression of relevant miRNAs may suggest the subject suspected of having the disease may be a candidate for MSC-based therapy, according to certain embodiments. Likewise, miRNA profiles that indicate limited changes in the differential miRNA expression profile may suggest that the subject suspected of having the disease may not be a candidate for MSC-based therapy, according to other embodiments.

### Pharmaceutical Compositions

Aspects of the current disclosure relate, in some cases, to a pharmaceutical composition to deliver one or more miRNAs from an MSC-EV-miRNA profile, wherein the pharmaceutical composition does not include intact MSCs or MSC-EVs. In some embodiments, the pharmaceutical composition comprises at least one naked miRNA (e.g., identified using an MSC-EV-miRNA profile). As used herein, “naked miRNA” refers to miRNA that is not complexed to another compound (e.g., is not encapsulated within a lipid nanoparticle. In some embodiments, the pharmaceutical composition comprises at least one miRNA (e.g., identified using an MSC-EV-miRNA profile) and a pharmaceutically acceptable excipient (e.g., carrier). As used herein, “pharmaceutically acceptable excipient” or “pharmaceutically acceptable carrier” refers to a pharmacologically inactive material used together with a pharmacologically active material to formulate the compositions. Pharmaceutically acceptable excipients comprise a variety of materials known in the art, including but not limited to saccharides (such as glucose, lactose, and the like), preservatives such as antimicrobial agents, reconstitution aids, colorants, saline (such as phosphate buffered saline), and buffers.

In some embodiments, the pharmaceutical composition comprises at least one miRNA (identified using an MSC-EV-miRNA profile) and a delivery vehicle, e.g., a lipid nanoparticle, encapsulating the at least one miRNA. In some cases, the pharmaceutical composition comprises greater than or equal to 1 miRNAs, greater than or equal to 2 miRNAs, greater than or equal to 3 miRNAs, greater than or equal to 5 miRNAs, greater than or equal to 7 miRNAs, greater than or equal to 10 miRNAs, greater than or equal to 12 miRNAs, greater than or equal to 14 miRNAs, greater than or equal to 16 miRNAs, or greater than or equal to 20 miRNAs. In other embodiments, the pharmaceutical composition comprises less than or equal to 20 miRNAs, less than or equal to 16 miRNAs, less than or equal to 12 miRNAs, less than or equal to 10 miRNAs, less than or equal to 7 miRNAs, less than or equal to 5 miRNAs, less than or equal to 3 miRNAs, or less than or equal to 1 miRNA.

The at least one miRNA may comprise hsa-miR-7107-5p, hsa-miR-6803-5p, hsa-miR-6798-5p, hsa-miR-760, hsa-miR-6727-5p, hsa-miR-4763-3p, hsa-miR-3652, hsa-miR-885-3p, hsa-miR-766-3p, hsa-miR-3175, hsa-miR-6893-5p, hsa-miR-6875-5p, hsa-miR-6799-5p, hsa-miR-6787-5p, or any combination thereof.

In some embodiments, the delivery vehicle may comprise a lipid nanoparticle and/or a liposome. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5:505-10). However, compositions that have different structures in solution than the normal vesicular structure are also encompassed. For example, the lipids may assume a micellar structure or merely exist as nonuniform aggregates of lipid molecules.

The at least one miRNA may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, or lipid/nucleic acid compositions are not limited to any particular structure in solution. For example, they may be present in a bilayer structure, as micelles, or with a “collapsed” structure. They may also simply be interspersed in a solution, possibly forming aggregates that are not uniform in size or shape.

In another embodiment, the liposome comprises a transfection reagent (e.g., a cationic and/or anionic lipid). Liposomes, in another embodiment, increase intracellular stability, increase uptake efficiency and improve biological activity. In another embodiment, liposomes are hollow spherical vesicles composed of lipids arranged in a similar fashion as those lipids which make up the cell membrane. In some embodiments, the liposomes comprise an internal aqueous space for entrapping water-soluble compounds. In another embodiment, liposomes can deliver the at least one MSC-EV-miRNA to cells in an active form.

In one embodiment, the composition comprises a lipid nanoparticle (LNP) and at least one miRNA.

The term “lipid nanoparticle” (LNP) refers to a particle having at least one dimension on the order of nanometers (e.g., 1-1000 nm) which includes one or more lipids. In some embodiments, LNPs comprise at least one agent that is either organized within inverse lipid micelles and encased within a lipid monolayer envelope or intercalated between adjacent lipid bilayers (e.g., lipid bilayer-agent-lipid bilayer). In some embodiments, the morphology of the LNPs is not like a traditional liposome, which are characterized by a lipid bilayer surrounding an aqueous core, as they possess an electron-dense core, where the cationic/ionizable lipids are organized into inverted micelles around the encapsulated agent.

In some embodiments, the lipid nanoparticles are substantially non-toxic. In certain embodiments, the at least one agent, when present in the lipid nanoparticles, is resistant in aqueous solution to degradation by intra- or intercellular enzymes

The LNP may comprise any lipid capable of forming a particle to which the at least one miRNA is attached, or in which the at least one miRNA is encapsulated or complexed. The term “lipid” refers to a group of organic compounds that are derivatives of fatty acids (e.g., esters) and are generally characterized by being insoluble in water but soluble in many organic solvents. Exemplary lipids are shown elsewhere herein.

In one embodiment, the LNP comprises one or more cationic lipids and one or more stabilizing lipids. Stabilizing lipids include neutral lipids, anionic lipids and pegylated lipids.

In one embodiment, the LNP comprises a cationic lipid. As used herein, the term “cationic or ionizable lipid” refers to a lipid that is cationic or becomes cationic (protonated) as the pH is lowered below the pKa of the ionizable group of the lipid, but is progressively more neutral at higher pH values. At pH values below the pKa, the lipid is then able to associate with negatively charged nucleic acids. In certain embodiments, the cationic lipid comprises a zwitterionic lipid that assumes a positive charge on pH decrease.

In various embodiments, the LNP comprises a cationic or ionizable lipids, stabilizing lipids, sterol, and a lipid-anchored polyethylene glycol (i.e., PEGylated lipids).

In certain embodiments, the LNP comprises one or more stabilizing lipids (e.g. neutral or anionic lipids) which help to encapsulate the cargo and stabilize the formation of particles during their formation.

In various embodiments, the LNPs further comprise a steroid or a steroid analogue.

In certain embodiments, the LNP comprises one or more targeting moieties that targets the LNP to a cell or cell population. For example, in one embodiment, the targeting domain is a ligand which directs the LNP to a receptor found on a cell surface. Exemplary targeting domains include but are not limited to toll like receptors or other damage or pathogen associated molecular receptors.

In certain embodiments, LNPs are formed by co-infusing an aqueous solution of mRNA and an ethanolic solution of lipid through a microfluidic device resulting in spontaneous vesicle formation.

In certain embodiments, the LNP comprises one or more internalization domains. For example, in one embodiment, the LNP comprises one or more domains which bind to a cell to induce the internalization of the LNP. For example, in one embodiment, the one or more internalization domains bind to a receptor found on a cell surface to induce receptor-mediated uptake of the LNP. In certain embodiments, the LNP is capable of binding a biomolecule in vivo, where the LNP-bound biomolecule can then be recognized by a cell-surface receptor to induce internalization. For example, in one embodiment, the LNP binds systemic ApoE, which leads to the uptake of the LNP and associated cargo.

Other exemplary LNPs and their manufacture are described in the art, for example in U.S. Patent Application Publication No. US20120276209, Semple et al., 2010, Nat Biotechnol., 28 (2): 172-176; Akinc et al., 2010, Mol Ther., 18 (7): 1357-1364; Basha et al., 2011, Mol Ther, 19 (12): 2186-2200; Leung et al., 2012, J Phys Chem C Nanomater Interfaces, 116 (34): 18440-18450; Lee et al., 2012, Int J Cancer., 131 (5): E781-90; Belliveau et al., 2012, Mol Ther nucleic Acids, 1: e37; Jayaraman et al., 2012, Angew Chem Int Ed Engl., 51 (34): 8529-8533; Mui et al., 2013, Mol Ther Nucleic Acids. 2, e139; Maier et al., 2013, Mol Ther., 21 (8): 1570-1578; and Tam et al., 2013, Nanomedicine, 9 (5): 665-74, each of which are incorporated by reference in their entirety.

### Engineered MSCs for Cell Therapy

Aspects of the current disclosure relate, in some cases, to a cell therapy comprising genetically engineered MSC cells configured to over-express and package one or more miRNAs, identified using a MSC-EV-miRNA profile, into EVs. In certain embodiments, the cell therapy comprises the engineered MSCs (i.e., MSCs and EVs); however, in some cases, secreted EVs may be isolated and used to directly deliver the miRNAs of interest to a subject (e.g., similar to LNPs).

In some embodiments, the MSCs may be genetically engineered to overexpress hsa-miR-7107-5p, hsa-miR-6803-5p, hsa-miR-6798-5p, hsa-miR-760, hsa-miR-6727-5p, hsa-miR-4763-3p, hsa-miR-3652, hsa-miR-885-3p, hsa-miR-766-3p, hsa-miR-3175, hsa-miR-6893-5p, hsa-miR-6875-5p, hsa-miR-6799-5p, hsa-miR-6787-5p, or any combination thereof. In other embodiments, the MSCs may be genetically engineered to overexpress hsa-miR-760, hsa-miR-3175, hsa-miR-885-3p, has-miR-766-3p, or any combination thereof. Other miRNAs (and combinations thereof) are also possible.

In some embodiments, a cell therapy comprises a culture of MSCs that overexpress a single miRNA of interest, such as a miRNA identified from an MSC-EV-miRNA profile. In certain embodiments, the cell therapy comprises a mixture of subpopulations of MSCs, wherein each subpopulation overexpresses a single miRNA of interest. The cell therapy may also comprise one or more subpopulations of MSCs engineered to overexpress multiple miRNAs (see below).

Aspects of the current disclosure relate, in some cases, to a method of producing therapeutic MSCs, wherein the therapeutic MSCs are genetically engineered MSC cells configured to over-express one or more miRNAs identified from a MSC-EV-miRNA profile In some embodiments, the method comprises obtaining a first biological sample from a healthy first subject and a second biological sample from a subject suspected of having a disease (e.g., cancer, ARDS, myocardial infarction). In other embodiments, the method comprises adding the first biological sample and the second biological sample to a first culture of MSCs and a second culture of MSCs, respectively, and detecting whether at least one MSC-EV-miRNA is present in the first and/or second culture of MSCs using, for example, next generation sequencing (e.g., microRNA-Seq). As described elsewhere herein, a series of analyses may be done following miRNA sequencing (e.g., functional prediction, overlap analyses, and discriminant analysis) to create a differential EV-miRNA profile based on the presence or absence of each miRNA, relative to the first health subject (or other appropriate control). miRNAs overexpressed in the MSC-EV-miRNA profile may then be used as therapeutic targets for overexpression in clinical grade MSCs.

MSCs may be engineered to overexpress a miRNA of interest using any technique known to those of ordinary skill in the art, such as, for example, infection with a virus that carries the gene of interest (e.g., a recombinant gene) or by direct transfer of a plasmid DNA that carries the gene of interest (e.g., a recombinant gene). For example, any miRNA of interest, e.g., hsa-miR-760, may be cloned, for example, into a commercially available lentivector system (e.g., XMIRXpress cloning lentivector, System Biosciences) comprising an RNA sequence tag, e.g., XMotif, that targets small RNAs to exosomes for packaging. In some embodiments, the engineered MSCs may indefinitely express the recombinant gene of interest (i.e., acts like a cell line), whereas in some instances the engineered MSCs may only transiently express the recombinant gene of interest.

In some embodiments, the method comprises engineering MSCs to overexpress more than one gene of interest. Any technique known to those of ordinary skill may be used to engineer MSCs that overexpress and package multiple genes of interest into EVs. For example, in some cases, different expression vectors may be used, each carrying a different miRNA gene of interest. In certain cases, a single vector may be constructed containing multiple genes each with its own promoter. Some additional options may include using a translational fusion approach, wherein two genes of interest are genetically joined in frame, which may ensure stoichiometric production of both miRNAs. Another strategy may include using internal ribosome entry sites (IRES), which facilitate ribosome binding to the second and subsequent transcription units. Other strategies and approaches are also possible. The method, according to certain embodiments, further comprises delivering the therapeutic MSCs to a subject in need thereof (e.g., subjects diagnosed with ARDS).

Primers for Rapid Detection of miRNAs Profiles

Aspects of the current disclosure relate, in some cases, to an oligonucleotide primer mix. The primer mix may be used, for example, for the quantification of miRNA expression in MSC-EVs (i.e., a biomarker for QC) to ensure the correct combinations of miRNAs are being generated (e.g., miRNAs identified in the MSC-EV-miRNA profile). In some cases, the primer mix comprises a forward primer and/or a reverse primer. In some embodiments, the oligonucleotide primer mix comprises a stem-loop reverse transcriptase (stem-loop RT) primer and/or a linear primer. In certain embodiments, the primer mix comprises one or more oligonucleotides, wherein each oligonucleotide is configured to hybridize to a single miRNA of interest, wherein the miRNA of interest is selected from the group consisting of hsa-miR-7107-5p, hsa-miR-6803-5p, hsa-miR-6798-5p, hsa-miR-760, hsa-miR-6727-5p, hsa-miR-4763-3p, hsa-miR-3652, hsa-miR-885-3p, hsa-miR-766-3p, hsa-miR-3175, hsa-miR-6893-5p, hsa-miR-6875-5p, hsa-miR-6799-5p, hsa-miR-6787-5p.

In some embodiments, an oligonucleotide primer mix comprising one or more oligonucleotide primers, may be used to detect the presence (or absence) of a miRNA of interest in a sample (e.g., during quality control check for MSC cell therapy). For example, in some embodiments, a stem-loop hairpin RT may be used to bind to the target miRNA at a 3′ end and then reversed transcribed using a reverse transcriptase (e.g., MultiScribe reverse transcriptase) and the RT products quantified using conventional PCR (e.g., Taqman PCR) that includes a miRNA-specific forward primer, a reverse primer, and a dye-labeled probe (e.g., a Taqman probe).

Quantitative RT-PCR using DNA primers is another example for which a primer mix may be used to quantify miRNAs of interest in a sample. Without wishing to be bound by theory, this method relies on poly(A) tailing of the miRNAs followed by reverse transcription (RT) with a tagged poly(T) primer. The RT products may be subsequently quantified using standard PCR with a primer set (e.g., a forward primer and a reverse primer) that is specific for a target miRNA transcript (e.g., 5′ tag and the 3′ tag).

In certain embodiments, the number of primers in a primer mix may be greater than or equal to 1 primer, greater than or equal to 2 primers, greater than or equal to 4 primers, greater than or equal to 6 primers, greater than or equal to 8 primers, greater than or equal to 10 primers, greater than or equal to 12 primers, greater than or equal to 14 primers, greater than or equal to 20 primers, or more. In other embodiments, the number of primers in the primer mix is less than or equal to 20 primers, less than or equal to 14 primers, less than or equal to 12 primers, less than or equal to 10 primers, less than or equal to 10 primers, less than or equal to 8 primers, less than or equal to 4 primers, less than or equal to 2 primers, less than or equal to 2 primers, less than or equal to 1 primer, etc.

### Primer Kit

Aspects of the current disclosure relate, in some cases, to a kit for detecting the presence of at least one miRNA, identified using a MSC-EV-miRNA profile, in a biological sample. In some embodiments, the kit comprises a primer mix comprising one or more oligonucleotides, wherein each oligonucleotide is configured to hybridize to a single miRNA of interest, wherein the miRNA of interest is selected from the group consisting of hsa-miR-7107-5p, hsa-miR-6803-5p, hsa-miR-6798-5p, hsa-miR-760, hsa-miR-6727-5p, hsa-miR-4763-3p, hsa-miR-3652, hsa-miR-885-3p, hsa-miR-766-3p, hsa-miR-3175, hsa-miR-6893-5p, hsa-miR-6875-5p, hsa-miR-6799-5p, hsa-miR-6787-5p. The kit may also comprise, according to certain embodiments, a reagent (e.g., a fluorescent probe) for performing a nucleic acid assay (e.g., PCR) to detect the at least one MSC-EV-miRNAs using the nucleic acid pair, and instructions for performing the assay to detect the at least one MSC-EV-miRNAs. The primer mix, according to some embodiments, comprises between 1 and 14 primer pairs for detecting up to 14 different miRNAs. In other embodiments, the primer mix comprises between 1 and 28 primers for detecting between 1 and 14 miRNAs of interest. In some embodiments, the kit may be used to screen cultures of MSCs (intended for cell therapy applications) for the presence of therapeutic miRNAs (identified via an MSC-EV-miRNA profile) for the treatment of ARDS and other inflammatory diseases (e.g., COPD, stroke, myocardial infarction).

### MSC Cell Source

The current disclosure relates, in some aspects, to obtaining mesenchymal stromal/stem cells (MSCs). In some embodiments, the MSCs are of a human origin. In some embodiments, the MSCs are of an animal origin (e.g., dog, cat, or monkey). Primary MSCs may be obtained from any suitable source (i.e., tissue), such as for example, bone marrow (BM), adipose tissue (AD), and/or placental membranes/umbilical cord blood (UC) and cultured using, for example, a cell culture device. In some embodiments, the culture of MSCs may comprise BM-MSCs; in some cases, the culture of MSCs may comprise AD-MSCs. In other embodiments, the culture of MSCs may comprise UC-MSCs. MSCs may also be obtained from differentiation of various progenitor cells, such as embryonic stem cells, induced pluripotent stem cells, and the like. Combinations are also possible (e.g., a culture may comprise 50% BM-MSCs and 50% AD-MSCs, where the percentage is relative to the total number of MSCs in the culture).

In some embodiments, a culture of MSCs comprise BM-MSCs. In some cases, the BM-MSCs comprise greater than or equal to 10%, greater than or equal to 25%, greater than or equal to 50%, greater than or equal to 75%, greater than or equal to 100% of the total number of MSCs in a culture. In other embodiments, the BM-MSCs comprise less than or equal to 100%, less than or equal to 75%, less than or equal to 50%, less than or equal to 25%, less than or equal to 10%, of the total number of MSCs in a culture.

In some embodiments, a culture of MSCs comprises AD-MSCs. In some cases, AD-MSCs comprise greater than or equal to 10%, greater than or equal to 25%, greater than or equal to 50%, greater than or equal to 75%, greater than or equal to 100% of the total number of MSCs in a culture. In other embodiments, the AD-MSCs comprise less than or equal to 100%, less than or equal to 75%, less than or equal to 50%, less than or equal to 25%, less than or equal to 10% of the total number of MSCs in a culture.

In some embodiments, a culture of MSCs comprises UC-MSCs. In some cases, UC-MSCs comprise greater than or equal to 10%, greater than or equal to 25%, greater than or equal to 50%, greater than or equal to 75%, greater than or equal to 100% of the total number of MSCs in a culture. In other embodiments, the UC-MSCs comprise less than or equal to 100%, less than or equal to 75%, less than or equal to 50%, less than or equal to 25%, less than or equal to 10% of the total number of MSCs in a culture.

Those of ordinary skill may understand that the confluency of a culture of MSCs may negatively affect EV production. Therefore, the culture of MSCs, according to some embodiments, may be grown to greater than or equal to 25% confluency, greater than or equal to 50% confluency, or greater than or equal to 75% confluency. In certain embodiments, the culture of MSCs may be grown to less than or equal to 75% confluency, less than or equal to 50% confluency, or less than or equal to 25% confluency.

It is also generally understood that the number of cell passages (i.e., replicative senescence) of a culture of MSCs may also negatively affect MSC EV production. Therefore, the culture of MSCs, according to some embodiments, may be passage between 3 and 8 times before exhibiting morphological abnormalities. In some embodiments, the MSCs may be passaged greater than or equal to 3 times, greater than or equal to 4 times, greater than or equal to 5 times, greater than or equal to 6 times, greater than or equal to 7 times, or greater than or equal to 8 times. In other embodiments, the MSCs may be passaged less than or equal to 8 times, less than or equal to 7 times, less than or equal to 6 times, less than or equal to 5 times, less than or equal to 4 times, or less than or equal to 3 times.

In certain cases, a MSC cell may be an immortalized MSC line. Immortalized MSCs may be used, for example, to ensure batch reproducibility, to avoid inter-individual donor variability, and to maintain bioactivity during culture expansion. Any technique known to those of skill in the art may be used to immortalize the MSCs. For example, in some embodiments, an embryonic stem cell-derived MSC may be immortalized by transfection of a lentivirus carrying the c-Myc oncogene.

The current disclosure relates, in some aspects, to culturing mesenchymal stromal/stem cells (MSCs). MSCs may be cultured using any technique known to those of skill in the art, such as for example, a bioreactor, or other in vitro cell culture device (e.g., culture dishes, multilayered cell culture flasks, hollow fiber bioreactors, stirred-tank bioreactors, and spheroidal aggregates of MSCs). In some embodiments, culturing MSCs using 3D systems (e.g., bioreactors) increases EV production between 40-fold to 100-fold, compared with 2D culture systems (e.g., culture dish). In some cases, culturing MSCs using the 3D systems increases EV production by greater than or equal to 40-fold, greater than or equal to 60-fold, greater than or equal to 80-fold, or greater than or equal to 100-fold, compared with the 2D culture system. In other embodiments, culturing MSCs using the 3D systems increases EV production by less than or equal to 100-fold, less than or equal to 80-fold, less than or equal to 60-fold, or less than or equal to 40-fold, compared with the 2D culture system.

In some embodiments, a culture of MSCs may be grown in a cell growth media. In some cases, the cell growth media comprises a defined cell growth media. In certain embodiments, the defined growth media is xeno-free and/or EV-free, for example, for use in clinical applications where MSC source variability and animal contaminations are of concern. In other embodiments, the cell growth media comprises an undefined media, comprising for example, human serum or human platelet lysate. Human platelet lysate comprises a plurality of growth factors, cytokines, hormones, proteins, carbohydrates, lipids that stimulate cell proliferation and is often used as a substitute for fetal bovine serum.

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

In order that the invention described herein may be more fully understood, the following examples are set forth. The examples described in this application are offered to illustrate the compounds, pharmaceutical compositions, and methods provided herein and are not to be construed in any way as limiting their scope.

### Example 1. Extracellular Vesicle (EV) Characteristics Following Bronchoalveolar Lavage Fluid (BALF) Exposure

Extracellular vesicles (EVs) derived from mesenchymal stromal cells (MSCs) may be used as a therapeutic for acute respiratory distress syndrome (ARDS). As described herein, MSC gene and protein expression are modulated by the ARDS lung environment. The effect of this environment on MSC-EV characterization and miRNA content was investigated, as described below.

BALF Exposure does not Significantly Affect Number or Size Distribution, but Decreases CD63 Expression in hMSC-Secreted EVs

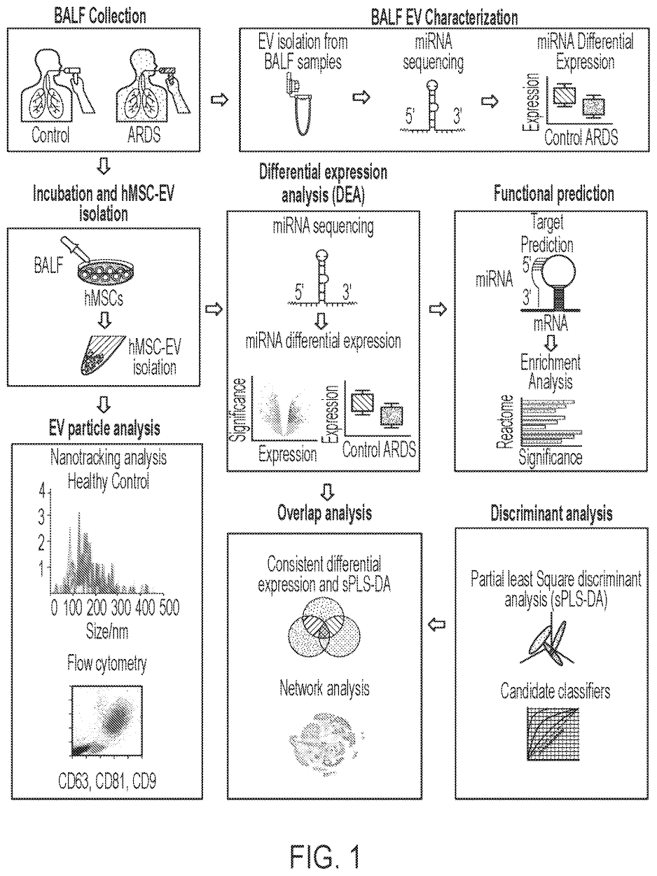

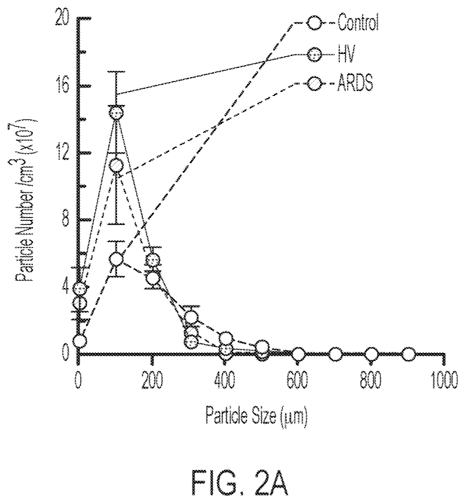

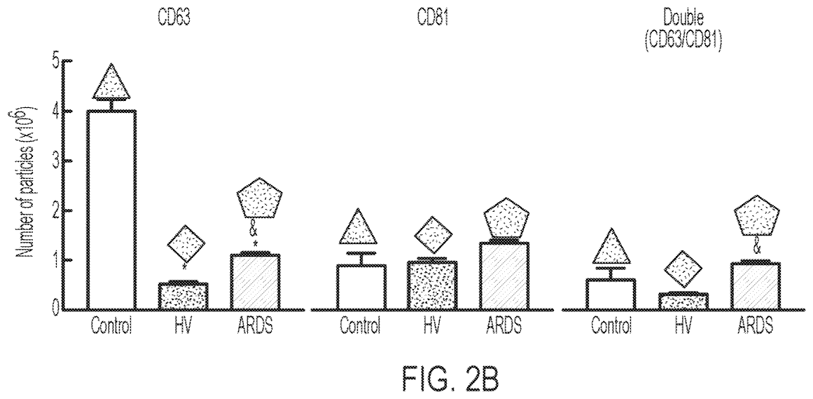

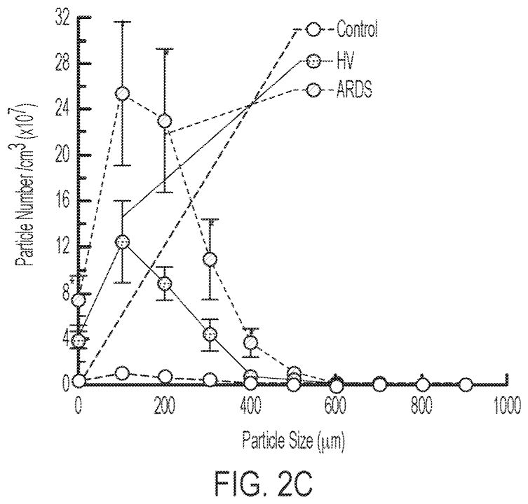

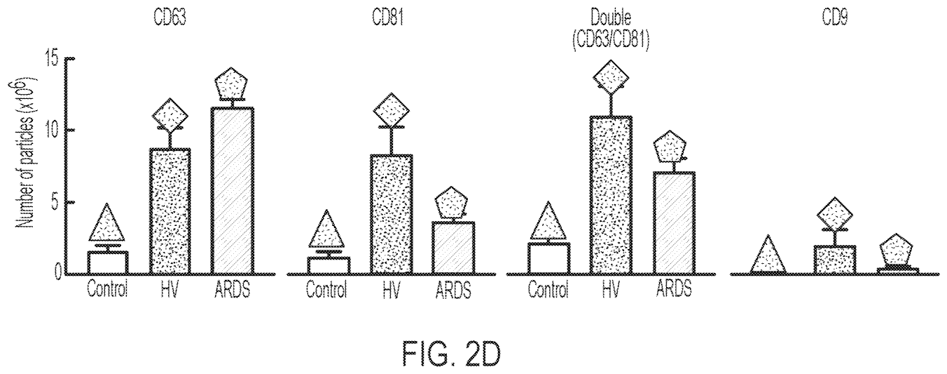

An overall schematic of the studies is presented in FIG. 1, summarizing the experimental protocols and analytical approaches utilized. Initially, to determine if exposure of hMSCs to different lung environments alters EV number and size, using BALF as a clinical surrogate, hMSC-EVs were prepared from hMSCs exposed to BALF from ARDS patients or HVs, or to control medium. NTA analyses revealed that, independent of whether the cells were exposed to control medium or BALF, 90% of hMSC-secreted EVs were between 50-200 μm in size, and the number of EVs was not significantly different (FIG. 2A). Both HV and ARDS BALF exposure decreased CD63 positive EVs overall compared to control, with HV BALF exposure resulted in lower CD63 expression than ARDS BALF exposure (FIG. 2B, left). There was no difference in EV CD81 expression between experimental conditions. The difference in CD63 expression between the HV and ARDS conditions translated into a significant difference in double positive (CD63/CD81) EV expression (FIG. 2B, middle and right). No CD9 expression was detected (data not shown).

EV Number, but not Tetraspanin Expression is Different in EVs Derived Directly from ARDS Versus HV BALF Samples

Whereas EVs isolated from BALF-exposed hMSCs showed a narrow size distribution and similar area under the curve (AUC) measurements, the size distribution of EVs isolated from BALF samples alone encompassed a wider range, with about 20% of the EVs from both ARDS and HVs being larger than 200 μm. Furthermore, ARDS BALF samples had significantly more particles than HV or control samples (FIG. 2C). Flow imaging revealed two major findings that stood in contrast to the analyses of EVs isolated from hMSCs. Specifically, there was an increase in CD63 and CD81 expression in both HV and ARDS BALF compared to control. Additionally, there was detectable CD9 expression in both HV and ARDS BALF EVs compared to lack of expression in EVs derived from hMSCs exposed to either HV or ARDS BALF (FIG. 2D).

### Example 2. EVs Derived from Resting Control hMSCs Contain miRNAs Predicted to Down Regulate Inflammatory Pathways

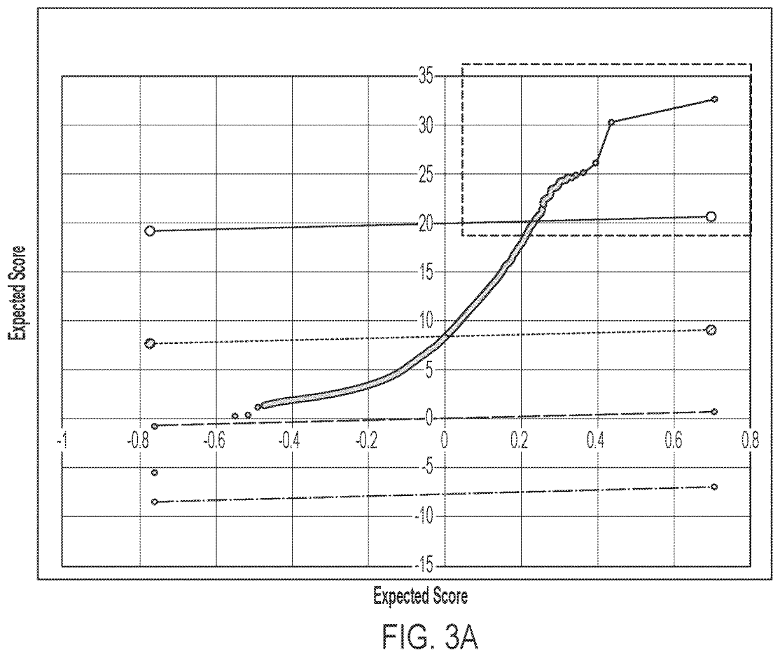

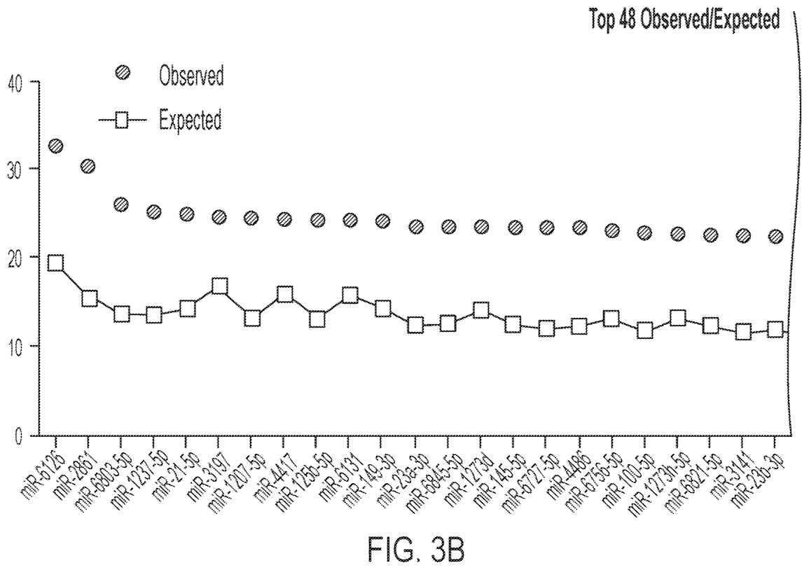

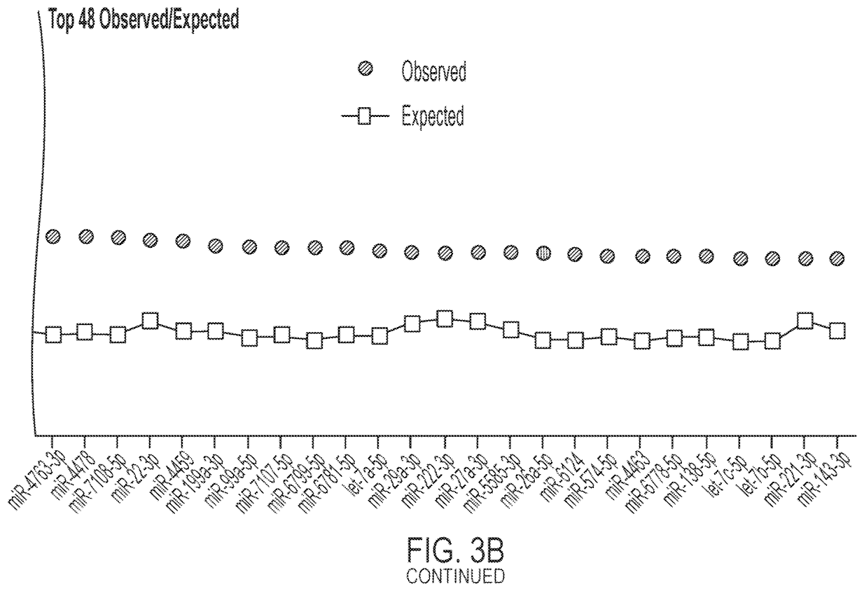

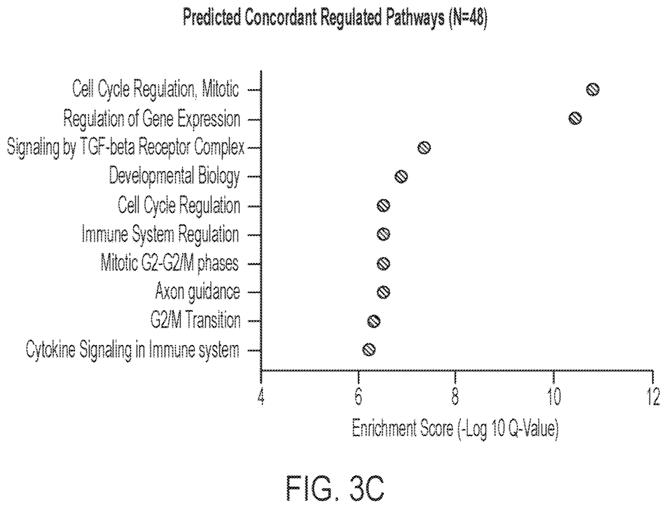

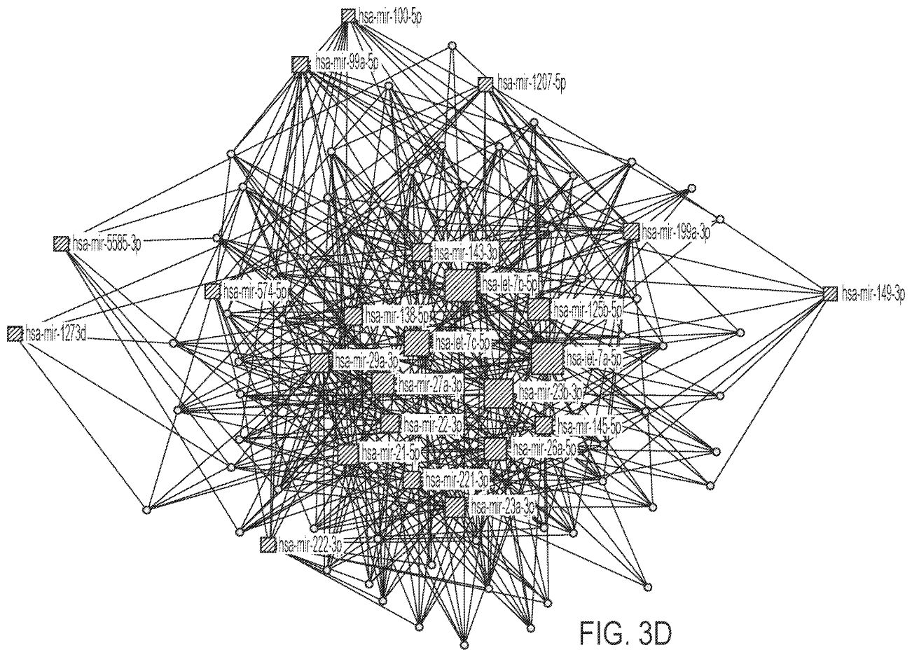

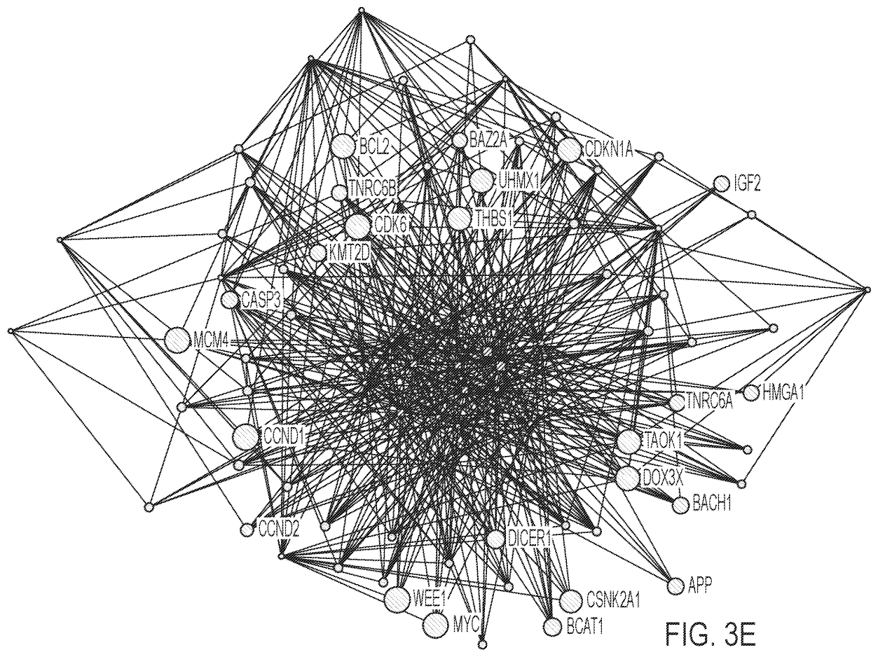

Before investigating the effect of the inflammatory lung environment on EV miRNA cargo, miRNAs were profiled in EV preparations obtained from control hMSCs (exposed to serum-free medium only). A total of 770 miRNAs were found to be present, of which 48 were markedly over-represented (observed/expected) as defined by a count number significantly above the average count of all miRNAs across all biological replicates (significant analysis of microarray [SAM] score ≥20, delta [δ] was set at 7 (default), False Discovery Rate [FDR]=0.0, FIGS. 3A and 3B). Enrichment analysis for these 48 miRNAs was performed in miRNet based on target prediction (DIANA miRTarbase v8.0) and identified over-representation of miRNAs regulating genes involved in cell cycle, regulation of gene expression, immune system regulation, immune system cytokine signaling, and Transforming Growth Factor β (TGFβ) receptor complex signaling among other pathways (FIG. 3C). Network analysis was used to visualize predicted ‘direct’ interactions between the top 48 over-represented miRNAs and experimentally identified gene targets. The top network-predicted miRNAs and gene targets were those involved in the regulation of cell cycle (FIGS. 3D and 3E).

### Example 3. MiRNAs in hMSC-EVs are not Randomly Packaged and can be Altered by Environmental Factors

This experiment examined whether exposure of hMSCs to BALF from different environments alters EV miRNA cargo. EVs were isolated from either conditioned medium from hMSCs exposed to serum-free medium (control, N=16), BALF from ARDS patients (ARDS, N=12), or healthy volunteers (HV, N=14). The expression level of 2,083 miRNAs contained in EVs isolated from these 42 samples was profiled using miRNA whole transcriptome sequencing. Principal component analysis (PCA) of normalized unfiltered miRNA counts revealed clustering of samples by treatment assignment, suggesting miRNA content was not randomly packaged and could be altered by exposure of hMSCs to different environmental milieu.

### Example 4. HMSCs Differentially Package miRNAs into EVs in Response to Different Inflammatory Environments

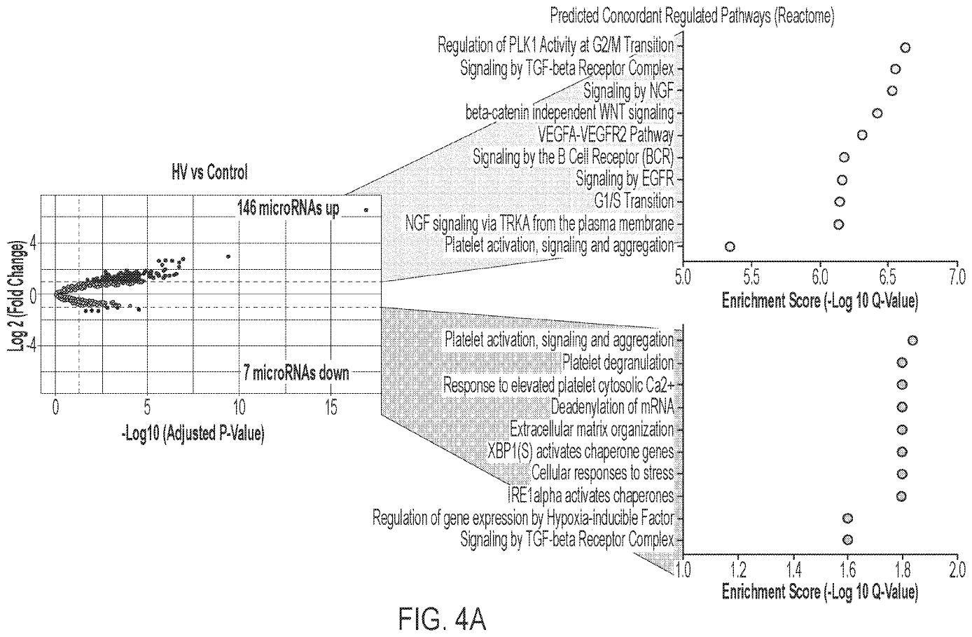

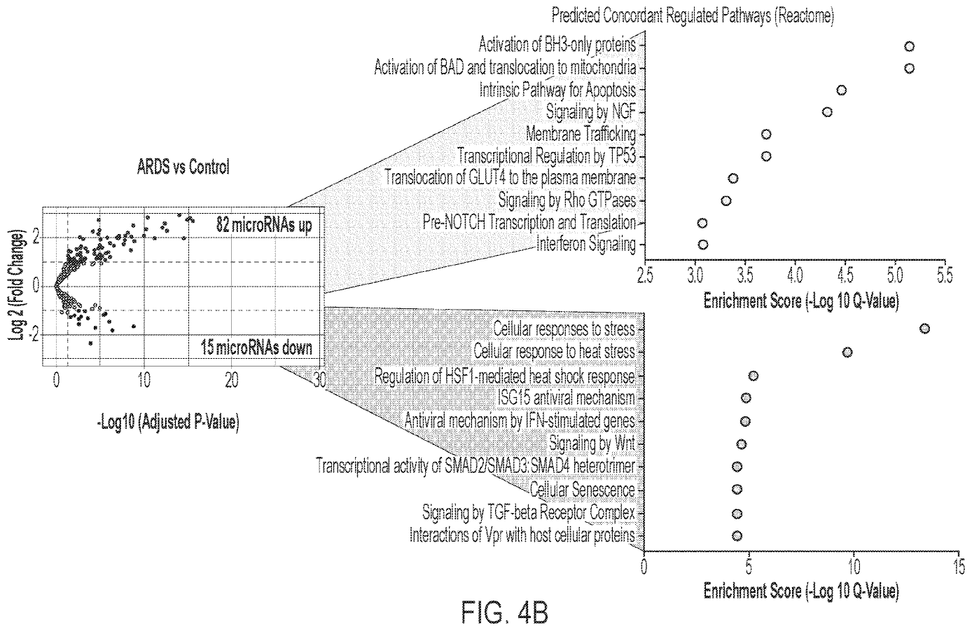

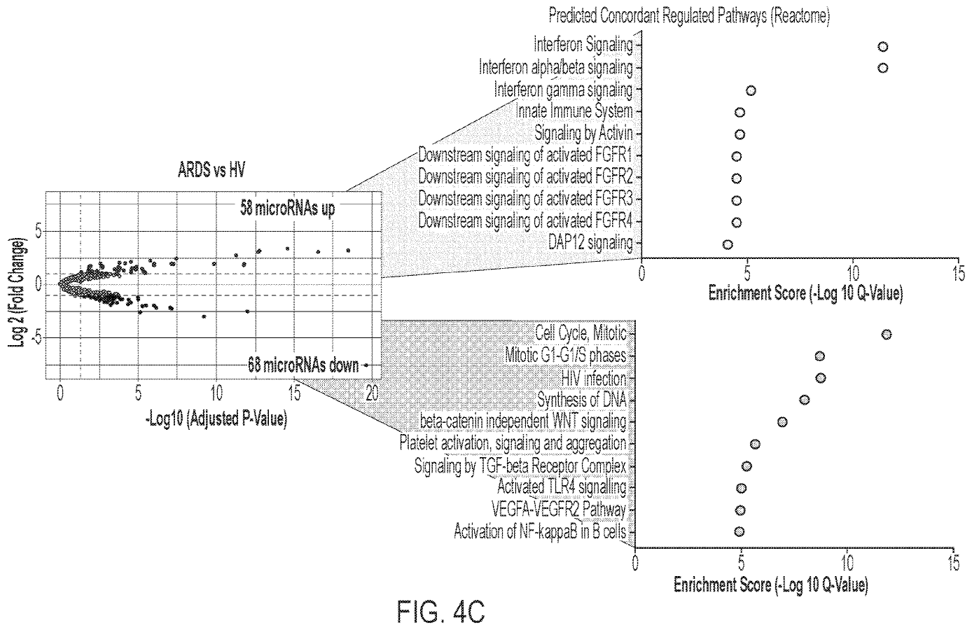

To determine the effect of different inflammatory environments on EV miRNA cargo, three pairwise comparisons were performed: HV vs control (DE=153 miRNAs), ARDS vs control (DE=97 DE miRNAs), and ARDS vs HV (DE=126 miRNAs). MiRNAs were deemed to be differentially expressed if they met statistical cut-offs of more than a two-fold change in expression (Log 2 fold change [FC]) and a FDR≤0.05. Volcano plots in FIGS. 4A-4C show over- and under-represented miRNAs for each comparison. The top 10 pathways predicted to be concordantly regulated for each comparison are shown in FIGS. 4A-4C. EVs from hMSCs exposed to HV BALF had more miRNAs associated with decreased expression of genes involved in the cell cycle and in TGFβ, VEGF, and EGFR signaling compared to controls. (FIG. 4A). In contrast, EVs from hMSCs exposed to HV BALF had fewer miRNAs involved in cellular responses to stress, including extracellular matrix organization and activation of hypoxia inducible factor (HIF), compared to the controls. Some groups, such as genes involved in platelet activation, signaling and aggregations had individual genes both over- and under-represented. Compared to control, EVs from hMSCs exposed to ARDS BALF, had an over-representation of miRNAs predicted to inhibit genes involved in cellular response to stress and interferon signaling, and an under-representation of miRNAs predicted to enhance the expression of genes involved in BH3 (e.g. Bax and Bad) selective triggering of canonical mitochondrial apoptosis in response to developmental cues, or stress-signals like DNA damages (FIG. 4B). Comparing miRNAs that were differentially expressed in ARDS vs HV EVs, it was surprising that the number of under-represented miRNAs is greater than the previous comparisons of control vs either HV or ARDS, respectively. The affected genes include those involved in cell cycle, DNA synthesis, TLR4, and NF-kappa B activation after exposure to ARDS BALF. In contrast, miRNAs that are over-represented in ARDS-exposed hMSCs compared to HV were enriched for those that regulate genes involved in interferon, activin (related to TGF-β), and FGFR signaling (FIG. 4C).

Overlap Analysis Demonstrates Consistent Differential Expression of EV miRNAs Between Experimental Groups

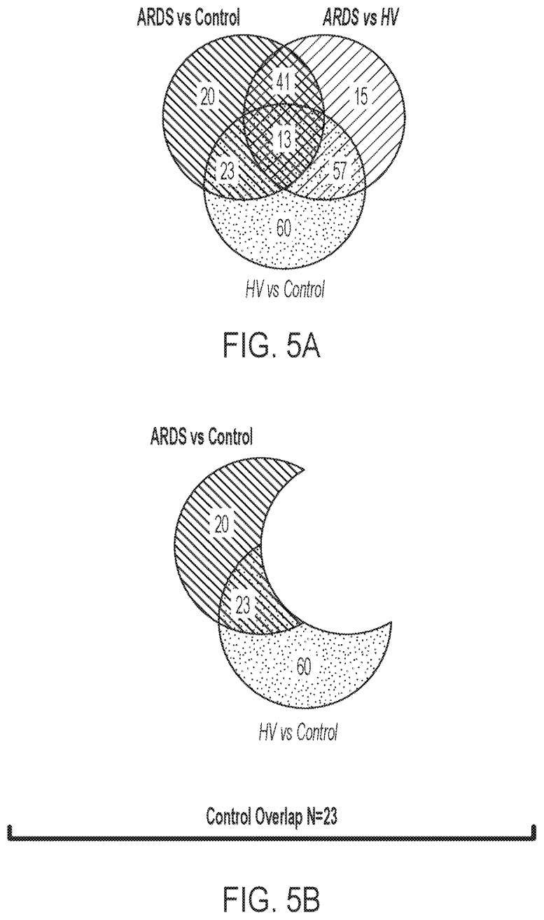

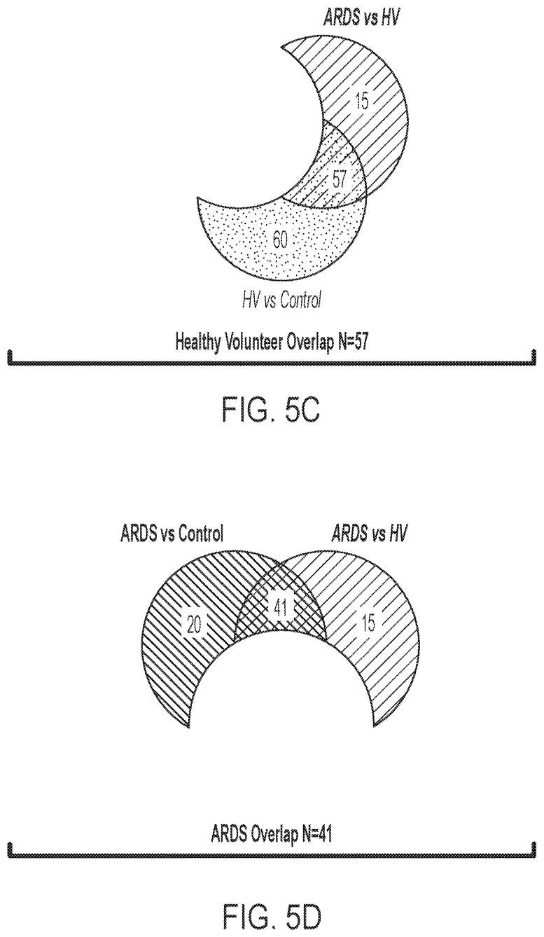

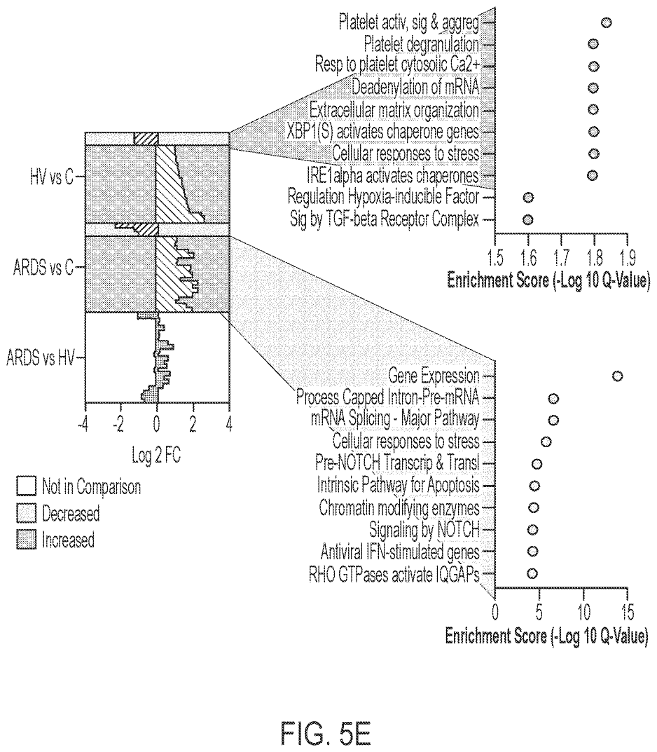

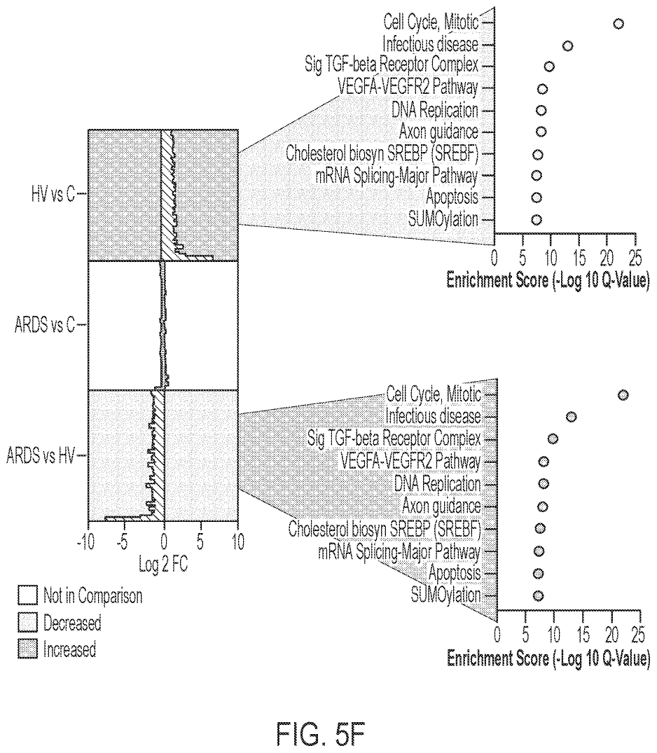

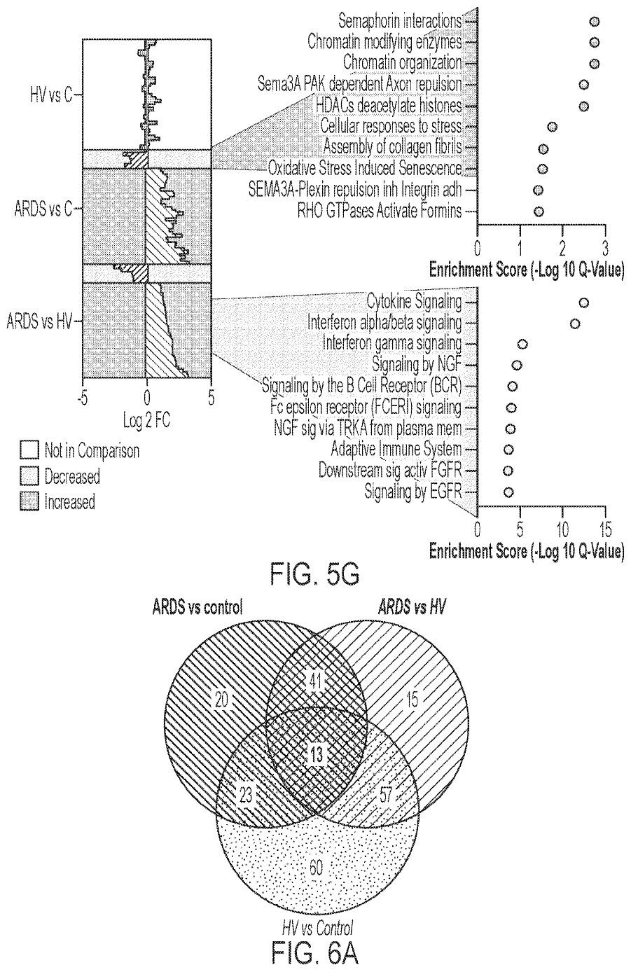

Venn diagrams were used to visualize the overlap between lists of miRNAs differentially regulated for each comparison (FIGS. 5A-5D). Consistent differential expression across more than one comparison was used to filter miRNAs that were more likely to be altered by each of the three conditions (i.e., control versus exposure to BALF from HV or ARDS patients, FIGS. 4B-4C). Patterns of miRNA expression were visualized by plotting the Log 2 FC in miRNA expression for each overlapped miRNA across each of the comparisons (FIGS. 5E-5G). Enrichment analyses for miRNAs in each overlap analysis are shown in FIGS. 5E-5G. Functional analysis showed miRNAs are differentially expressed in all comparisons with control hMSC-EVs (N=23) predicted to be involved in the regulation of gene expression, cell stress, RNA processing, alternative splicing, and TGF-β receptor complex signaling (the top 10 pathways enriched in the analysis are shown in FIG. 5E). MiRNAs regulated in all comparisons with the HV BALF-exposed samples (N=57) were of particular interest because they were regulated in opposite direction in EVs from hMSCs exposed ARDS BALF. Depending on the direction of change of the miRNA, these miRNAs were predicted to increase or decrease pathways involved in cell cycle, infectious disease, as well as signaling by TGF-β regulation among others (FIG. 5F). MiRNAs responsive to ARDS BALF were predicted to be involved in cytokine signaling, interferon β and a signaling, and programmed cell death among others (FIG. 5G).

### Example 5. Identification of 52 miRNAs that May be Highly Associated with Biological and Therapeutic Effects of hMSCs

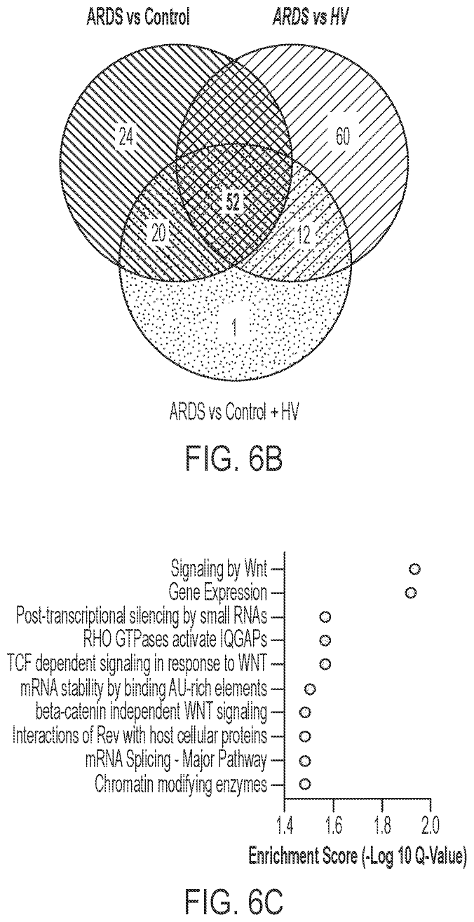

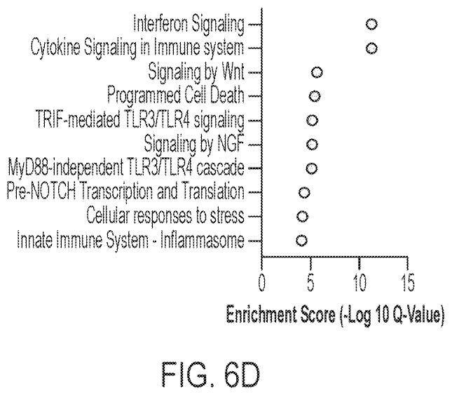

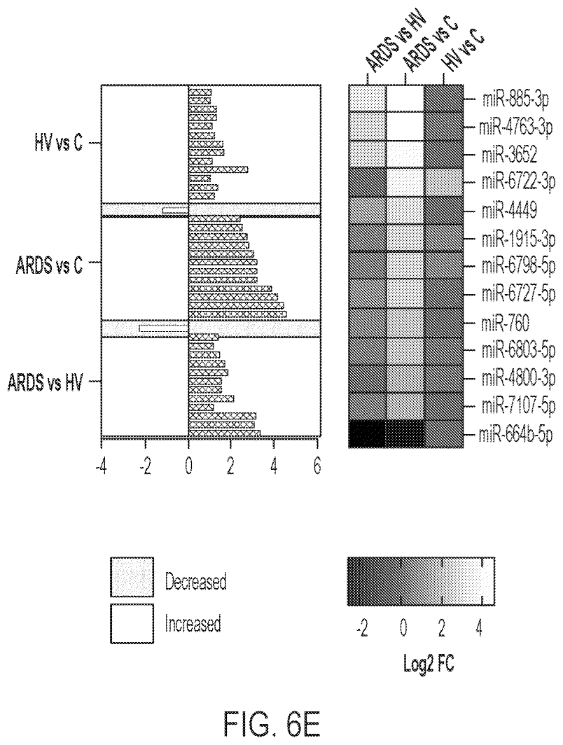

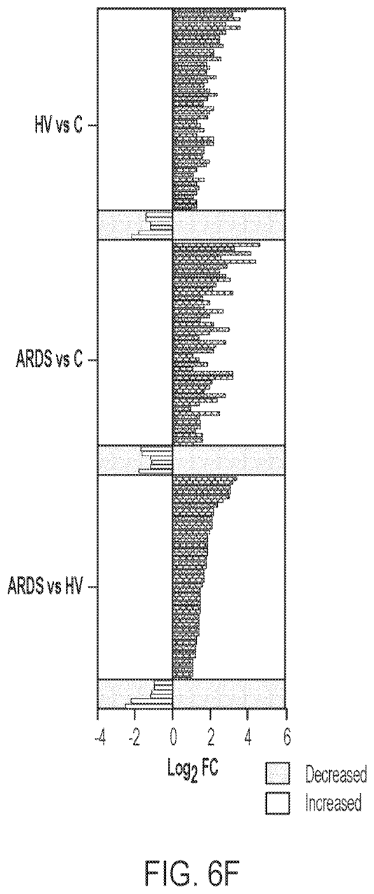

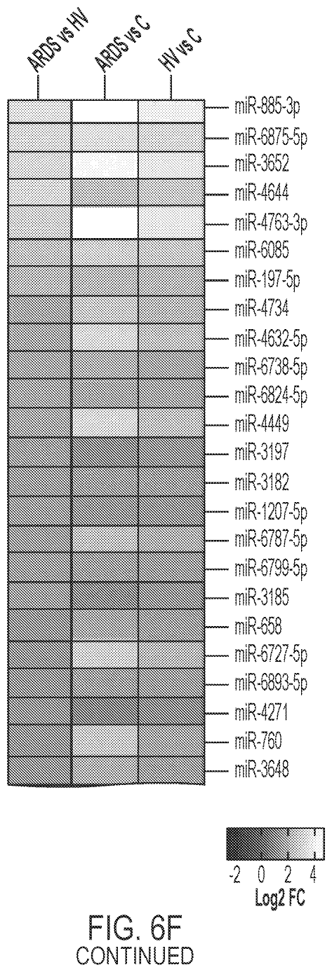

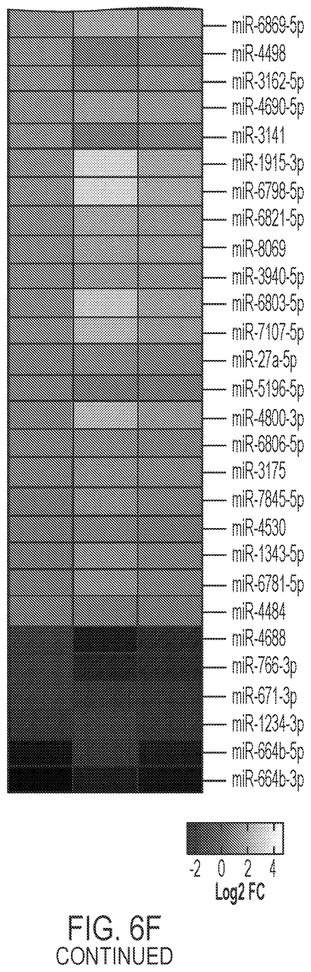

It was considered that miRNAs that were differentially present in all of the comparisons could be of biological and/or therapeutic significance since they appeared to be packaged inside an hMSC-derived EV in all experimental conditions. Accordingly, if hMSCs conferred a beneficial effect—these miRNAs were likely to be mechanistically involved in conferring such an effect rather than being non-specifically found inside an hMSC-derived EV. The overlap between the 3 pairwise comparisons demonstrated that 13 miRNAs were differentially regulated in all hMSC-EV preparations (FIG. 6A). To underscore the effects of ARDS-BALF on miRNA differential packaging, a separate pairwise comparison was performed, combining the effect of control and HV (control+HV). A total of 52 miRNAs were found to be robustly differentially expressed in all three comparisons and includes the 13 overlapping miRNAs (FIG. 6B; Table 1).

Pathways predicted to be regulated by the 13 co-differentially expressed miRNAs from overlap 1 (FIG. 6A) affected genes involved in Wnt signaling and mRNA processing and stability among others (FIG. 6C). MiRNAs co-regulated in overlap 2 (52 miRNAs, FIG. 6B), interacted with genes known to be involved in interferon and cytokine signaling, Wnt, Toll like receptor (TLR3/4), and nerve growth factor (NGF) signaling, as well as in programmed cell death (FIG. 6D). Similar patterns in miRNA expression change in each comparison were visualized as horizontal bar graphs. Heat maps summarizing consistency of change in miRNA expression across all 42 biological samples are shown in FIGS. 6E and 6F.

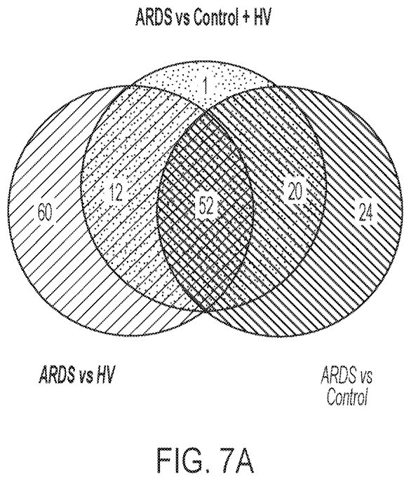

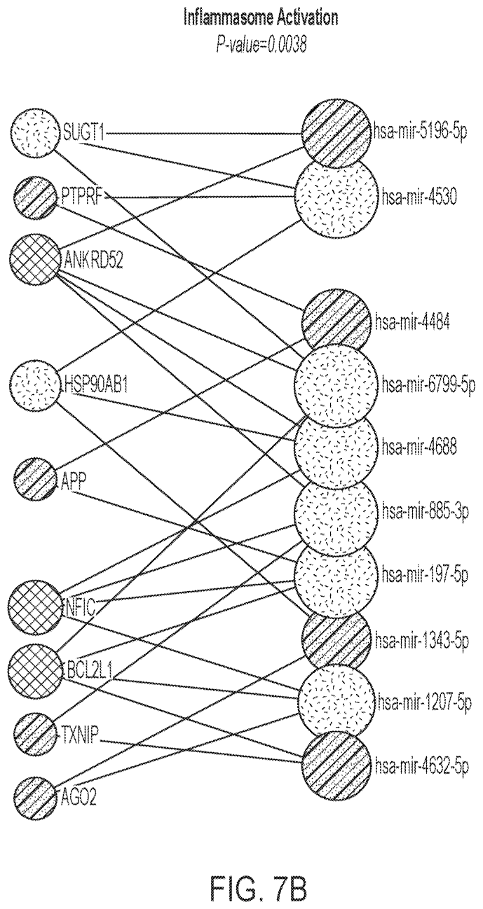

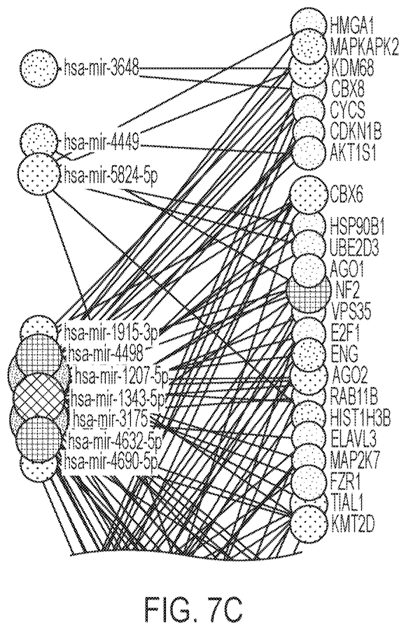

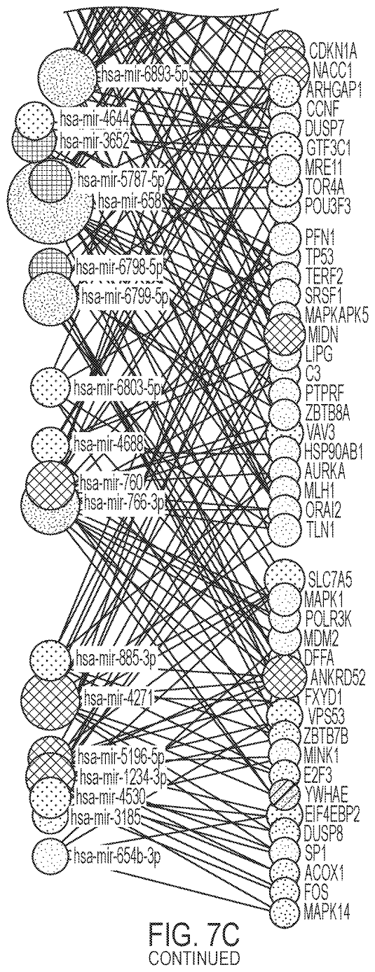

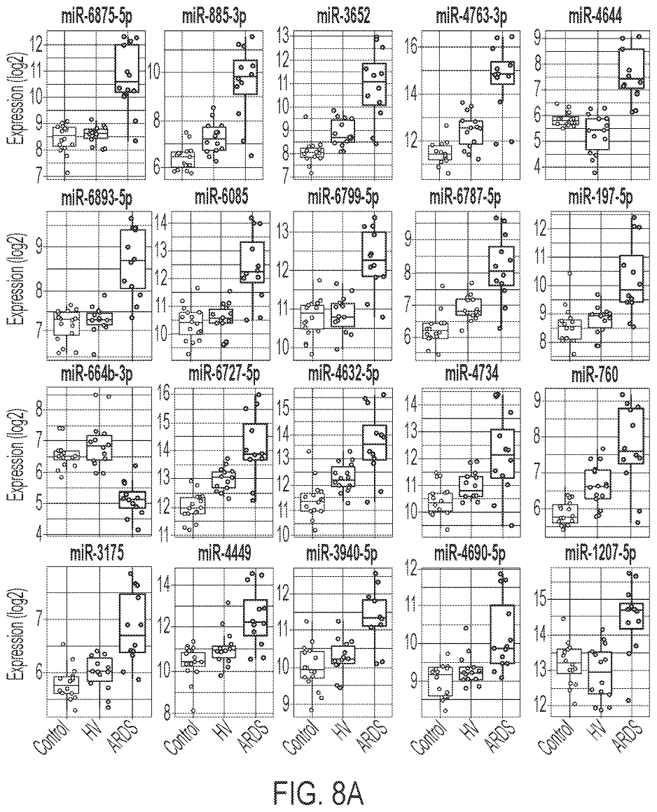

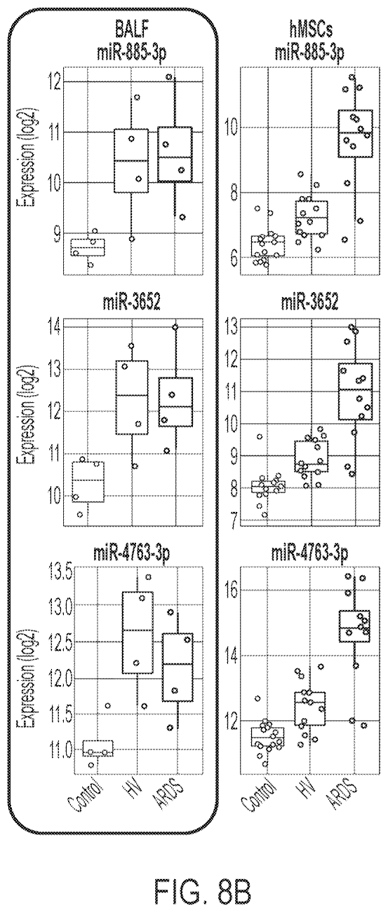

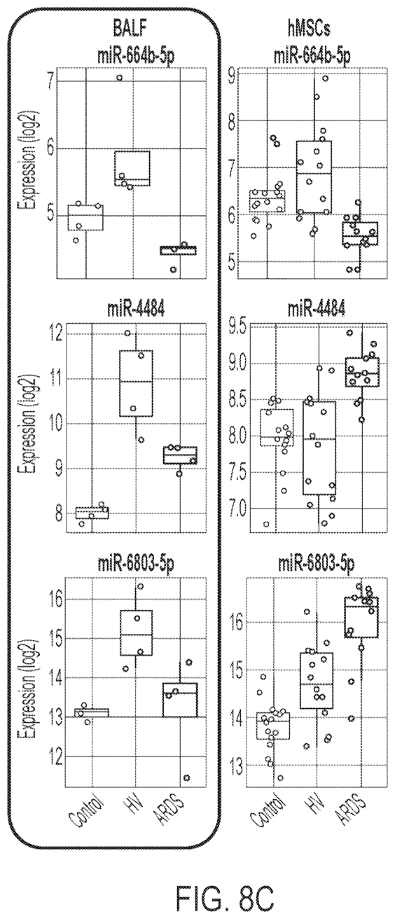

To identify miRNAs that were possibly associated with biological and therapeutic effects of hMSCs, miRNAs present in EVs were isolated from control hMSCs and differentially expressed across all treatment conditions; including demonstrated sustained differential expression when the effect of unstimulated and healthy volunteers (control+HV vs ARDS), were combined. Network analysis for overlap 2 miRNAs (N=52, FIG. 7A) showed that co-regulated miRNAs and their gene targets form functionally relevant interaction networks. Two of these networks, which are of particular interest in acute lung injury, involved inflammasome activation and cell-stress (FIGS. 7B and 7C). Sample level data is shown as box plots of the Log 2 normalized counts for the top 20 miRNAs from the two networks (ranked by FDR, FIG. 8A), demonstrated trends in differential expression of EV-derived miRNAs.

TABLE 1
Overlap analysis of LIMMA Output Combining HV + Control vs ARDS

| | | ARDS vs HV | | ARDS vs C | | ARDS vs [HV + C] | |
|---|---|---|---|---|---|---|---|
| miRNA | ID | Log2FC | FDR | Log2FC | FDR | Log2FC | FDR |
| miR-664b-3p | MIMAT0022272 | −2.5 | 5.82E−10 | −1.8 | 1.99E−09 | −2.2 | 4.41E−11 |
| miR-664b-5p | MIMAT0022271 | −2.2 | 1.08E−06 | −1.2 | 4.16E−04 | −1.8 | 1.47E−05 |
| miR-1234-3p | MIMAT0005589 | −1.2 | 1.17E−05 | −1.1 | 1.02E−05 | −1.2 | 2.08E−07 |
| miR-671-3p | MIMAT0004819 | −1.1 | 9.02E−03 | −1.2 | 3.11E−03 | −1.2 | 6.46E−04 |
| miR-766-3p | MIMAT0003888 | −1 | 1.24E−03 | −1.6 | 2.59E−08 | −1.4 | 4.61E−07 |
| miR-4688 | MIMAT0019777 | −1 | 1.96E−02 | −1.7 | 1.41E−04 | −1.4 | 3.10E−04 |
| miR-4484 | MIMAT0019018 | 1 | 4.33E−03 | 1.1 | 1.55E−06 | 1 | 1.92E−04 |
| miR-6781-5p | MIMAT0027462 | 1.1 | 9.32E−03 | 1.6 | 8.21E−06 | 1.3 | 1.05E−04 |
| miR-1343-5p | MIMAT0027038 | 1.1 | 1.38E−02 | 1.6 | 1.70E−03 | 1.3 | 1.89E−03 |
| miR-4530 | MIMAT0019069 | 1.1 | 1.17E−02 | 1.2 | 3.21E−03 | 1.1 | 8.16E−04 |
| miR-7845-5p | MIMAT0030420 | 1.1 | 4.07E−02 | 1.5 | 1.44E−02 | 1.3 | 1.20E−02 |
| miR-3175 | MIMAT0015052 | 1.2 | 1.07E−04 | 1.5 | 4.80E−08 | 1.4 | 2.29E−09 |
| miR-6806-5p | MIMAT0027512 | 1.2 | 3.01E−04 | 1.4 | 2.09E−06 | 1.3 | 2.19E−07 |
| miR-4800-3p | MIMAT0019979 | 1.2 | 1.63E−02 | 2.5 | 6.00E−10 | 1.7 | 1.02E−04 |
| miR-5196-5p | MIMAT0021128 | 1.3 | 4.04E−04 | 1 | 3.83E−03 | 1.1 | 2.69E−04 |
| miR-27a-5p | MIMAT0004501 | 1.3 | 5.52E−03 | 1.4 | 2.17E−03 | 1.3 | 8.60E−04 |
| miR-7107-5p | MIMAT0028111 | 1.4 | 1.07E−03 | 2.4 | 3.11E−14 | 1.8 | 1.57E−07 |
| miR-6803-5p | MIMAT0027506 | 1.4 | 1.07E−03 | 2.8 | 4.44E−19 | 2 | 1.20E−07 |
| miR-3940-5p | MIMAT0019229 | 1.4 | 1.69E−05 | 1.7 | 2.46E−07 | 1.6 | 1.92E−08 |
| miR-8069 | MIMAT0030996 | 1.4 | 3.66E−04 | 2 | 4.80E−08 | 1.7 | 1.76E−07 |
| miR-6821-5p | MIMAT0027542 | 1.4 | 1.09E−03 | 2.1 | 3.26E−08 | 1.7 | 6.24E−07 |
| miR-6798-5p | MIMAT0027496 | 1.5 | 6.47E−04 | 3.2 | 2.77E−21 | 2.2 | 1.40E−07 |
| miR-1915-3p | MIMAT0007892 | 1.5 | 6.83E−03 | 3.2 | 2.31E−15 | 2.2 | 7.80E−06 |
| miR-3141 | MIMAT0015010 | 1.5 | 5.89E−05 | 1.1 | 9.54E−04 | 1.3 | 1.02E−05 |
| miR-4690-5p | MIMAT0019779 | 1.5 | 3.41E−04 | 1.9 | 2.46E−07 | 1.7 | 2.52E−08 |
| miR-3162-5p | MIMAT0015036 | 1.5 | 7.48E−03 | 1.4 | 3.14E−02 | 1.5 | 1.26E−02 |
| miR-4498 | MIMAT0019033 | 1.5 | 2.89E−04 | 1.1 | 5.41E−03 | 1.3 | 4.11E−05 |
| miR-6869-5p | MIMAT0027638 | 1.6 | 2.91E−04 | 2.2 | 2.47E−09 | 1.9 | 1.71E−07 |
| miR-3648 | MIMAT0018068 | 1.7 | 4.03E−03 | 2.3 | 2.42E−06 | 2 | 4.72E−05 |
| miR-760 | MIMAT0004957 | 1.7 | 3.37E−04 | 2.8 | 2.52E−15 | 2.2 | 1.82E−09 |
| miR-4271 | MIMAT0016901 | 1.7 | 9.02E−03 | 1.4 | 2.86E−02 | 1.6 | 1.20E−02 |
| miR-6893-5p | MIMAT0027686 | 1.8 | 9.93E−10 | 2 | 7.87E−13 | 1.9 | 7.26E−18 |
| miR-6727-5p | MIMAT0027355 | 1.8 | 1.73E−05 | 3 | 2.02E−15 | 2.4 | 4.41E−11 |
| miR-658 | MIMAT0003336 | 1.8 | 4.26E−03 | 2.2 | 1.53E−04 | 2 | 1.10E−04 |
| miR-3185 | MIMAT0015065 | 1.9 | 7.66E−06 | 1.5 | 3.87E−08 | 1.7 | 1.81E−07 |
| miR-6799-5p | MIMAT0027498 | 1.9 | 1.19E−08 | 2 | 5.28E−11 | 1.9 | 1.98E−14 |
| miR-6787-5p | MIMAT0027474 | 1.9 | 9.02E−07 | 2.7 | 2.58E−13 | 2.3 | 4.97E−13 |
| miR-1207-5p | MIMAT0005871 | 1.9 | 1.62E−05 | 1.7 | 2.95E−06 | 1.8 | 1.10E−07 |
| miR-3182 | MIMAT0015062 | 1.9 | 2.13E−03 | 2 | 7.99E−03 | 2 | 6.12E−04 |
| miR-3197 | MIMAT0015082 | 2 | 6.24E−05 | 1.6 | 1.43E−04 | 1.8 | 7.59E−05 |
| miR-4449 | MIMAT0018968 | 2.1 | 3.13E−04 | 3.2 | 1.85E−12 | 2.6 | 8.61E−09 |
| miR-6824-5p | MIMAT0027548 | 2.1 | 4.58E−05 | 2.1 | 2.60E−05 | 2.2 | 1.09E−06 |
| miR-6738-5p | MIMAT0027377 | 2.1 | 3.68E−04 | 2.3 | 2.05E−05 | 2.2 | 3.80E−06 |
| miR-4632-5p | MIMAT0022977 | 2.2 | 5.50E−06 | 3.1 | 3.44E−11 | 2.7 | 9.96E−11 |
| miR-4734 | MIMAT0019859 | 2.2 | 2.96E−05 | 2.8 | 4.59E−09 | 2.5 | 2.40E−10 |
| miR-197-5p | MIMAT0022691 | 2.4 | 3.76E−07 | 2.5 | 1.51E−07 | 2.5 | 3.59E−11 |
| miR-6085 | MIMAT0023710 | 2.7 | 4.44E−10 | 2.9 | 3.79E−12 | 2.8 | 5.00E−17 |
| miR-4763-3p | MIMAT0019913 | 3 | 4.42E−10 | 4.4 | 6.59E−28 | 3.6 | 1.17E−19 |
| miR-4644 | MIMAT0019704 | 3.1 | 4.16E−12 | 2.6 | 2.31E−15 | 2.8 | 3.08E−18 |
| miR-3652 | MIMAT0018072 | 3.1 | 9.22E−11 | 4.2 | 3.57E−21 | 3.6 | 2.19E−20 |
| miR-6875-5p | MIMAT0027650 | 3.2 | 2.48E−15 | 3.3 | 3.55E−17 | 3.2 | 3.27E−26 |
| miR-885-3p | MIMAT0004948 | 3.4 | 7.31E−12 | 4.6 | 1.82E−26 | 3.9 | 2.02E−22 |

### Example 6. Differential Expression of Top 20 miRNAs Contained in BALF-EVs Strongly Suggest Biological Effects of Exposures on hMSC EV miRNAs Rather than Cross Contamination from BALF Samples

miRNAs contained in EVs directly isolated from the BALF of HVs (N=4), ARDS patients (N=4), and from the serum-free medium (N=5) used to incubate the hMSCs during BALF exposure experiments were assessed to determine if hMSC EV-miRNA content might reflect carry over of EVs in the BALF that adhered to the hMSCs and were not washed away prior to collection of the BALF-exposed hMSC-EVs. EV and miRNA isolation, as well as miRNA sequencing, was performed as described for hMSC-EV studies. Differential expression analysis was performed in two separate ways, first differential expression was determined using DESeq2 and the list of miRNAs differentially expressed in BALF was compared with the list of miRNAs differentially expressed in EVs. Separately, given the small sample size, group medians were compared using Mann-Whitney testing.

Of the top 20 miRNAs of interest differentially expressed in EVs derived from ARDS BALF-exposed hMSCs (FIG. 8A), 13 miRNAs were not differentially expressed in EVs directly obtained from ARDS BALF compared to HV BALF. Of the 7 miRNAs that were differentially expressed in the BALF and differentially present in hMSC-derived EVs, 3 had opposite directions of change (FIGS. 8B-8C), suggesting different biologic effects of both HV and ARDS BALF exposures rather than cross contamination from EVs found in either the HV or ARDS BALF samples.

### Example 7. Supervised Classification Analysis Produces a Signature of Overlapping Discriminatory miRNAs

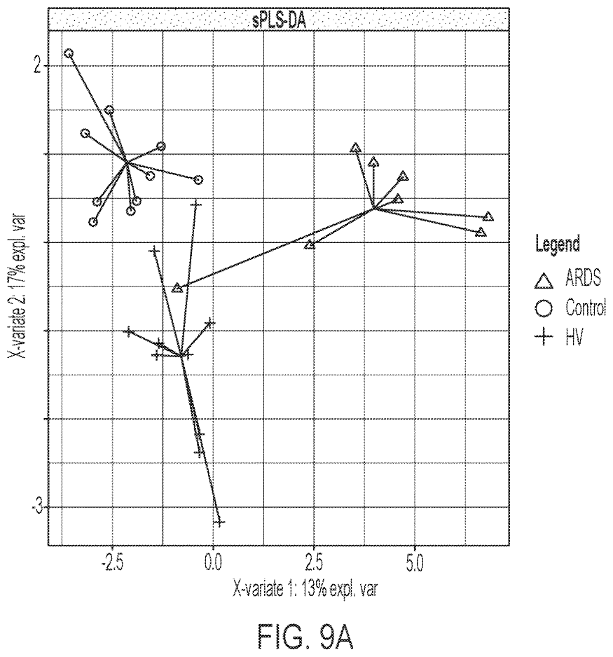

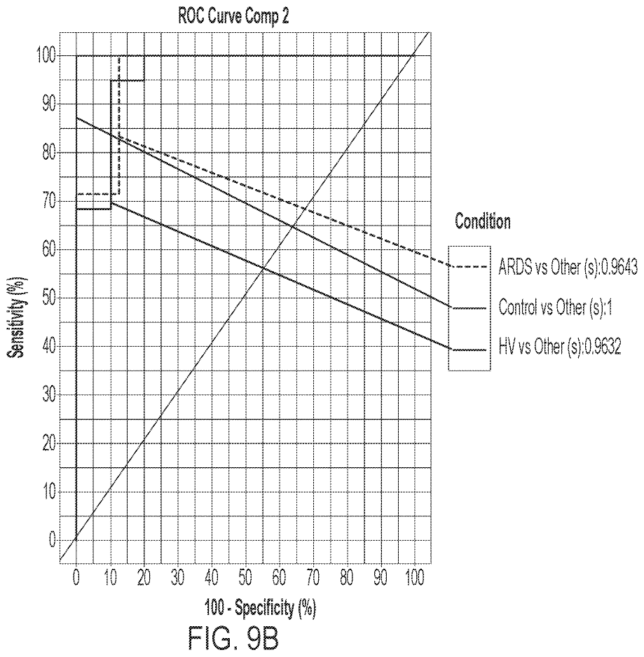

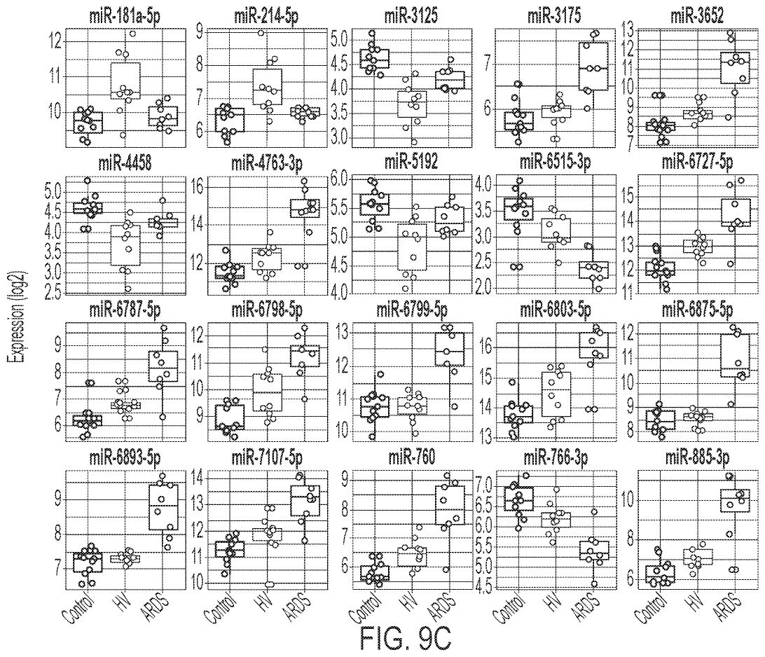

A discriminant analysis was used to determine whether HV or ARDS BALF-exposed and control hMSC-derived EVs could be classified using a reduced miRNA signature. Samples were split into training and test sets (70% versus 30%, respectively) and Sparse Partial Least Squares Discriminant Analysis (sPLS-DA) was used to develop the classification model on training data. A 2-component model with 15 and 5 miRNAs selected on the first two components, respectively, was found to have the lowest balanced discriminant error rate (0.0883). The 20-miRNA signature was found to discriminate samples into their appropriate treatment group (ARDS BALF-exposed, HV BALF-exposed, or control) with the first component separating ARDS from the other classes, and the second component providing resolution between HV and controls (FIG. 9A). The ability of the 20 miRNAs to accurately classify samples into treatment groups was validated using leave-one-out cross-validation (LOOCV) in the test cohort. The area under the receiver operating characteristic curve for ARDS versus HV+controls, which is based on prediction distances and averaged over all cross-validations, was 0.9643 (FIG. 9B). The accuracy of the signature in predicting each class for the test set was 0.92, 0.85, and 0.92 for ARDS, HV, and control groups, respectively.

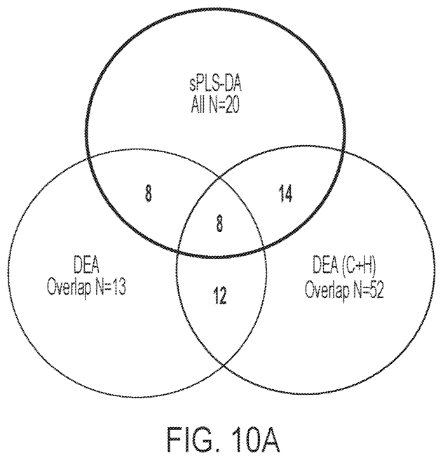

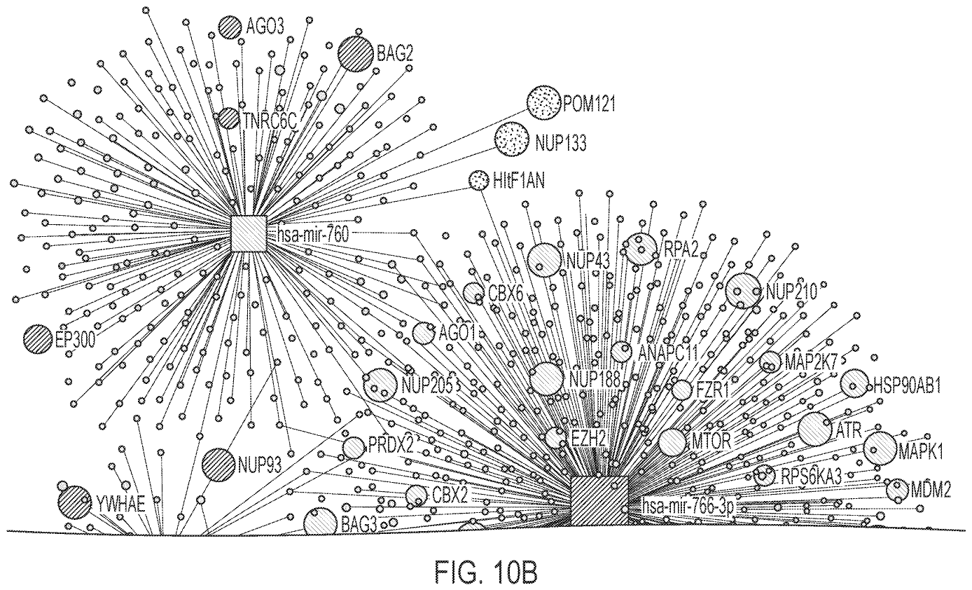

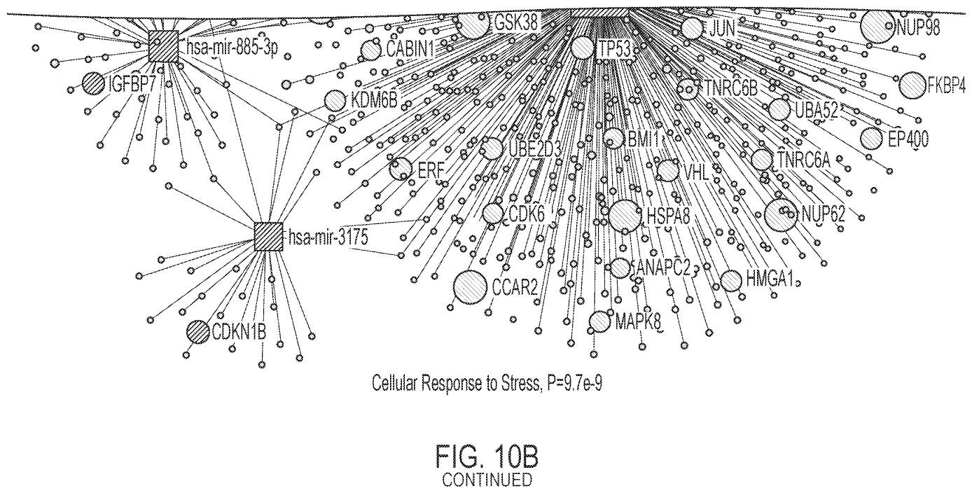

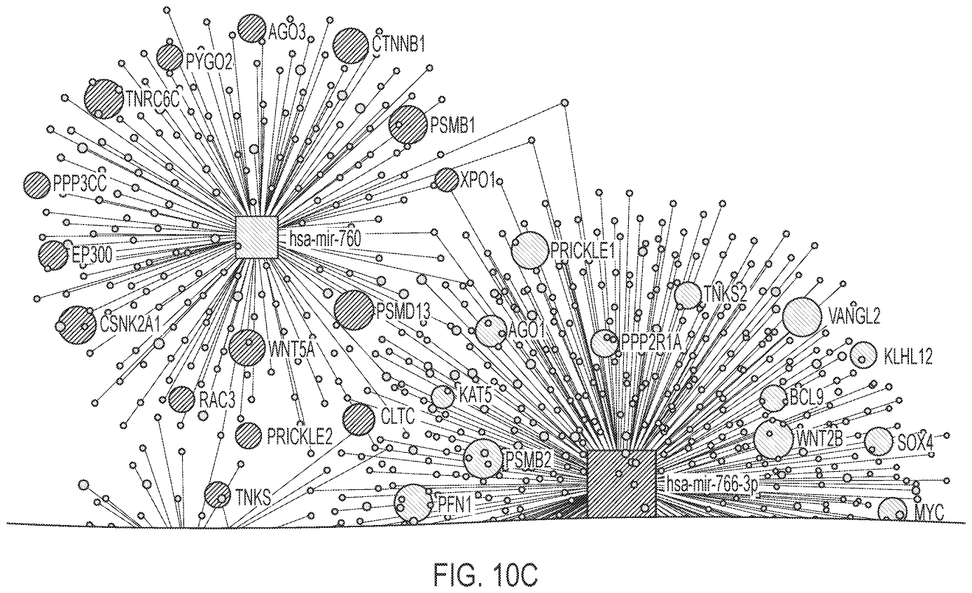

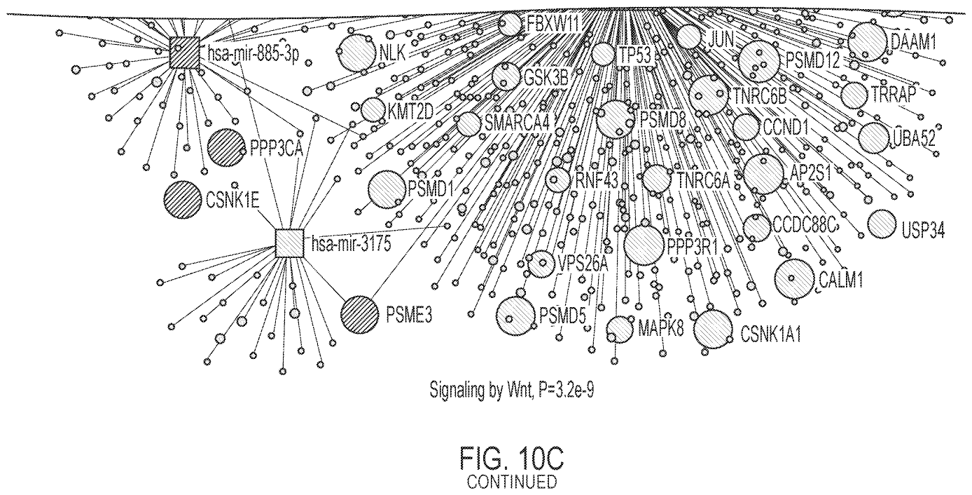

A total of 14 of the 20 classifiers identified using sPLS-DA were found to also be differentially expressed between treatment groups. Functional analysis and visual exploration of miRNA-target interactions in miRNet found that 4 of the 14 differentially expressed classifiers (miRNA-760, miRNA-3175, miRNA-855-3p, and miRNA-766-3p) form a putative in-silico regulatory network acting as putative “hub” regulators for 1259 putative targets of which 75 are involved in cellular responses to stress and in Wnt signaling (enrichment score=9.7E-9 and 3.2E-9, FIGS. 10B and 10C).

To understand how pathways predicted to be regulated by their gene targets could be implicated in acute lung injury a more detailed analysis of these 14 miRNAs was performed (Table 2). miRNA-766-3p shared direct links to genes involved in cell stress responses, whilst miRNA-885-3p, miRNA-3175, and miRNA-760 each contributed to the enrichment for genes involved in Wnt signaling (FIGS. 10A and 10B). MiRNA-885-3p had direct interaction with genes involved in pro-inflammatory cytokine regulation, apoptosis, chemoresistance, proliferation, and metastasis. MiRNA-766-3p regulated genes that inhibit inflammation by acting through NF-κB signaling. MiRNA-664b-3p, miRNA-4644, miRNA-6803-5p, miRNA-6869-5p, miRNA-3940-5p, and miRNA-766-3p were implicated in the regulation of proliferation. MiRNA-3175 promoted epithelial-mesenchymal transition by targeting Smad 7. MiRNA-760 was considered a possible tumor suppressor as it negatively regulated oncogenic proteins and decreased proliferation, cell cycle progression, migration, and differentiation. Summaries of miRNA level results and functional enrichment that may be relevant for acute lung injury for the top 14 differentially expressed miRNA classifiers and overlap miRNAs of interest are found in Table 2.

TABLE 2
Functional Predictions - Focus on Possible Roles in Acute Lung Injury

| | | | | | | | | Possible roles of importance in | |
|---|---|---|---|---|---|---|---|---|---|
| | | | | | | | | Acute Lung Injury | |
| | | | | | | | | Function based | |
| | | HV vs | | ARDS vs | | | | on Empirically | Function based on |
| | | Control | | Control | | ARDS vs HV | | confirmed | Computationally |
| MiRBase | MiRBase | | Adj | | Adj | | Adj | Targets | Predicted Targets |
| ID | Accession # | Log2FC | P-value | Log2FC | P-value | Log2FC | P-value | (TarBase v8.0) | (DIANA - Target Scan) |
| hsa-miR- | MIMAT0028111 | 1.03 | 3.50E−03 | 2.42 | 8.82E−13 | 1.39 | 2.33E−04 | No hits found on | No related GO category |
| 7107-5p | | | | | | | | TarBase v8.0 | found |
| hsa-miR- | MIMAT0027506 | 1.29 | 2.84E−04 | 2.74 | 1.20E−15 | 1.44 | 1.75E−04 | No hits found on | No related GO category |
| 6803-5p | | | | | | | | TarBase v8.0 | found |
| hsa-miR- | MIMAT0027496 | 1.62 | 1.66E−05 | 3.14 | 5.36E−19 | 1.52 | 1.48E−04 | No hits found on | No related GO category |
| 6798-5p | | | | | | | | TarBase v8.0 | found |
| hsa-miR- | MIMAT0004957 | 1.13 | 1.79E−03 | 2.79 | 5.68E−16 | 1.66 | 1.30E−05 | Regulates RHO | Predicted to regulate |
| 760 | | | | | | | | GTPases via IQ | integrity of adherens |
| | | | | | | | | motif-containing | junctions and the first |
| | | | | | | | | GTPase-activating | step of cholesterol, |
| | | | | | | | | proteins (IQGAPs) - | steroid hormones, and |
| | | | | | | | | scaffolding | vitamin D biosynthesis |
| | | | | | | | | proteins playing | (Lanosterol Synthase |
| | | | | | | | | central roles in | and 24- |
| | | | | | | | | cell-cell adhesion, | Dehydrocholesterol |
| | | | | | | | | polarity, and | Reductase) |
| | | | | | | | | motility. | |
| hsa-miR- | MIMAT0027355 | 1.20 | 8.04E−04 | 3.04 | 2.90E−19 | 1.84 | 9.51E−07 | No hits found on | Predicted |
| 6727-5p | | | | | | | | TarBase v8.0 | Glycosphingolipid |
| | | | | | | | | | biosynthesis (ST8 |
| | | | | | | | | | Alpha-N-Acetyl- |
| | | | | | | | | | Neuraminide Alpha-2,8- |
| | | | | | | | | | Sialyltransferase 5) and |
| | | | | | | | | | Phosphatidylinositol |
| | | | | | | | | | signaling system |
| | | | | | | | | | (PIP5K1C, PIK3R2 and |
| | | | | | | | | | PLCB2) |
| hsa-miR- | MIMAT0019913 | 1.36 | 1.18E−03 | 4.41 | 8.89E−30 | 3.06 | 2.00E−13 | No hits found on | Putative regulation of |
| 4763-3p | | | | | | | | TarBase v8.0 | GPX7 (Glutathione |
| | | | | | | | | | Peroxidase 7) involved |
| | | | | | | | | | in Glutathione |
| | | | | | | | | | metabolism and |
| | | | | | | | | | Detoxification of |
| | | | | | | | | | Reactive Oxygen |
| | | | | | | | | | Species. |
| hsa-miR- | MIMAT0018072 | 1.02 | 1.59E−02 | 4.15 | 3.33E−25 | 3.13 | 1.83E−13 | No hits found on | No related GO category |
| 3652 | | | | | | | | TarBase v8.0 | found |
| hsa-miR- | MIMAT0004948 | 1.20 | 5.52E−03 | 4.58 | 8.89E−30 | 3.37 | 2.78E−15 | Regulates | Putative regulation of |
| 885-3p | | | | | | | | SERPINE2 (serine | cell adhesion via |
| | | | | | | | | protease inhibitor | ICAM3 and NECTIN1 |
| | | | | | | | | e.g., Thrombin, | (organization of |
| | | | | | | | | urokinase, plasmin | adherens junctions and |
| | | | | | | | | and trypsin); | tight junctions in |
| | | | | | | | | regulates NFKB | epithelial and |
| | | | | | | | | Inhibitor Zeta; | endothelial cells) |
| | | | | | | | | TNFAIP3; and | |
| | | | | | | | | IRF3 | |
| hsa-miR- | MIMAT0003888 | −1 | 1.24E−03 | −1.6 | 2.59E−08 | −1.4 | 4.61E−07 | Anti-inflammatory | Predicted to regulate |
| 766-3p | | | | | | | | role by regulating | fatty acid synthase |
| | | | | | | | | complement 3 | (FASN) and Acetyl- |
| | | | | | | | | (C3), Jun, IRF3, | CoA carboxylase |
| | | | | | | | | STAT2, MAPK1, | (ACAC) - rate limiting |
| | | | | | | | | MAPK8, TBK1, | enzymes in the synthesis |
| | | | | | | | | TANK (inhibits | of fatty acids. |
| | | | | | | | | TRAF) | |
| hsa-miR- | MIMAT0015052 | N/C | N/C | 1.51 | 4.80E−08 | 1.2 | 1.07E−04 | Negative | Predicted to regulate |
| 3175 | | | | | | | | regulation of | vasopressin water |
| | | | | | | | | metabolic | resroption (Aqp2 and |
| | | | | | | | | processes and | VAMP2) and ECM- |
| | | | | | | | | DNA damage | receptor interaction |
| | | | | | | | | response dependent | (HSPG2, COL5A1, |
| | | | | | | | | upon p53 - may | COL1A1, COL5A3) |
| | | | | | | | | impact apoptosis in | |
| | | | | | | | | lung injury | |
| hsa-miR- | MIMAT0027686 | N/C | N/C | 1.98 | 2.77E−15 | 1.84 | 1.69E−12 | No hits found on | Predicted to affect |
| 6893-5p | | | | | | | | TarBase v8.0 | SNARE interactions in |
| | | | | | | | | | vesicular transport |
| hsa-miR- | MIMAT0027650 | N/C | N/C | 3.24 | 1.04E−21 | 3.15 | 3.45E−19 | No hits found on | Predicted to regulate |
| 6875-5p | | | | | | | | TarBase v8.0 | proteoglycans, Hyaluron |
| | | | | | | | | | (CD44), Heparan sulfate |
| | | | | | | | | | (SDC2) and fibroblast |
| | | | | | | | | | growth factor receptor 1 |
| | | | | | | | | | (FGFR1) important in |
| | | | | | | | | | wound healing, cell |
| | | | | | | | | | migration and repair. |
| hsa-miR- | MIMAT0027498 | N/C | N/C | 1.97 | 6.16E−12 | 1.92 | 1.45E−10 | No hits found on | Predicted to regulate |
| 6799-5p | | | | | | | | TarBase v8.0 | Von Willebrand Factor |
| | | | | | | | | | (vWF) critical for ECM |
| | | | | | | | | | interactions. HACD2 (3- |
| | | | | | | | | | Hydroxyacyl-CoA |
| | | | | | | | | | Dehydratase 2) in the |
| | | | | | | | | | conversion of long chain |
| | | | | | | | | | fatty acids to very long |
| | | | | | | | | | chain fatty acids. |
| | | | | | | | | | Pyridoxamine 5′- |
| | | | | | | | | | Phosphate Oxidase |
| | | | | | | | | | (PNPO) rate-limiting |
| | | | | | | | | | step in the synthesis of |
| | | | | | | | | | pyridoxal 5′-phosphate |
| | | | | | | | | | (vitamin B6) - required |
| | | | | | | | | | co-factor in both |
| | | | | | | | | | homocysteine |
| | | | | | | | | | metabolism and |
| | | | | | | | | | synthesis of |
| | | | | | | | | | neurotransmitters such |
| | | | | | | | | | as catecholamines. |
| hsa-miR- | MIMAT0027474 | N/C | N/C | 2.69 | 2.90E−16 | 1.93 | 5.31E−08 | No hits found on | Predicted to regulate |
| 6787-5p | | | | | | | | TarBase v8.0 | fatty acid biosynthesis |
| | | | | | | | | | and metabolism through |
| | | | | | | | | | regulation of fatty acid |
| | | | | | | | | | synthase (FASN), Acyl- |
| | | | | | | | | | CoA Synthetase Long |
| | | | | | | | | | Chain Family Member 3 |
| | | | | | | | | | (ACLS3) and Trans-2,3- |
| | | | | | | | | | Enoyl-CoA Reductase |
| | | | | | | | | | (TECR) |

### Example 8. Discussion

There remains a fundamental lack of knowledge as to the fate and actions of exogenously administered hMSCs and their EV products in clinical lung disease inflammatory environments. It was previously shown that the ARDS inflammatory environment, utilizing clinical BALF as a surrogate, has profound influence on hMSC gene and protein expression, recognizability by the host immune system, and on downstream effects on relevant immune effector cells. In some embodiments, it is shown that the ARDS inflammatory environment also influences hMSC-EV tetraspanin expression and the associated miRNA content. A data driven approach was used to identify the most abundant and consistently represented miRNAs in hMSC-EVs. Analysis of differential expression patterns found that EVs isolated from ARDS-exposed hMSCs contained more miRNAs predicted to inhibit genes involved in cellular response to stress and interferon signaling. In contrast, EVs isolated from hMSCs exposed to a healthy non-inflamed environment, contained more miRNAs predicted to inhibit genes involved in cell cycle and in TGF-β, VEGF, and EGFR signaling. Finally, an EV miRNA signature was identified and used to classify samples into treatment groups. In the future, these signatures might inform the biological activity of hMSCs and might be potency markers. Of the 14 miRNAs that passed both differential and discriminant analysis filters, 10 were mostly novel and very little data was available in the literature to elucidate their possible function. In contrast to empiric determinations, computational predictions suggest important roles in regulation of cell-cell, cell matrix, oxidative stress, fatty acid metabolism, programmed cell death, and endothelial cell activation (Table 1). From what is known about the remaining four (miRNA-760, miRNA-3175, miRNA-885-3p, and miRNA-766-3p), they notably formed an interaction network with their known target genes which was predicted to regulate acute lung critical processes—For example there were responses to cellular stress and Wnt signaling, strongly suggesting a biological role for miRNAs contained in hMSC-derived EVs.

EVs are increasingly recognized as mediating anti-inflammatory and other effects of their parent hMSCs. This includes pre-clinical models of acute lung injury and other lung diseases where EVs are as effective, sometimes more effective, than the parent EVs themselves. This provided a platform for initial clinical investigations of hMSC-derived EVs in patients with ARDS, bronchopulmonary dysplasia, and other lung diseases). However, the mechanisms by which hMSC-EVs can influence the inflammatory lung environment are still being elucidated. So far, the initial focus has been on miRNAs associated with the EVs. For example, miRNA-27 and its target gene VAV3 have been shown to play a role in cell infiltration and cell adhesion during acute lung injury.

In this study substantial and significant differences were observed in the profile of hMSC-EV associated miRNAs obtained under the different BALF exposure conditions. Interestingly, the number of isolated miRNAs decreased in ARDS-exposed hMSC-EVs compared to HV-exposed hMSC-EVs. One possible explanation is that disease results in loss of variability. Variability determines plasticity, i.e., the degree to which a gene can change its expression in response to environmental fluctuations. Under pathological stress, this plasticity may be lost. Another interesting determination was that hMSCs exposed to both ARDS and HV BALF samples produced EVs containing miRNAs with general anti-inflammatory activities such as inhibition of cell response to stress, and of IFN, TGF-β, VEGF, and EGFR signaling. Whereas in EVs isolated from control hMSCs exposed to serum-free medium, these pathways were predicted to be less affected. In addition, EVs isolated from hMSCs exposed to a healthy non-inflamed environment contained miRNAs that resulted in activation of proliferation, platelet activation, HIF signaling, and extracellular matrix—these are all pathways involved in wound healing and tissue remodeling.

In an attempt to identify key miRNAs, two different bioinformatic strategies, DEA and sPLS-DA, identified 14 miRNAs of interest. This list was further narrowed down by looking for miRNAs that worked together utilizing network analysis and visual exploration of miRNA-target interactions in miRNet. Using this strategy, 4 “hub” regulator miRNAs were identified: miRNA-760, miRNA-3175, miRNA-885-3p, and miRNA-766-3p, which are known to be involved in cellular response to stress and Wnt signaling. Interestingly, Wnt is an evolutionarily conserved pathway that regulates crucial aspects of cell fate determination, cell migration, cell polarity, neural patterning, and organogenesis during embryonic development. The Wnt/β-catenin pathway has been implicated in the induction, promotion, and abnormal repair of acute lung injury. One of strength of these observations is any functional predictions were limited to miRNA-mRNA interactions that have been demonstrated experimentally to occur based on a large experimental compendium of miRNA-mRNA interaction data.

Even though the hMSCs used in this study were washed carefully after BALF exposure, it was possible that some of the BALF-derived EVs were internalized or remained ‘stuck’ to the cells. The available literature on this is sparse and to address this, comparable analyses were performed on EVs isolated from the same BALF samples and from the serum-free media used to incubate the control hMSCs. Although overlapping miRNAs in the raw BALF samples as well as in the control media were present, a substantial number of significantly differentially expressed miRNAs were identified. For example, miRNA-885-3p, miRNA3652, and miRNA-4763-3p were not found to be differentially expressed between the raw BALF samples and control medium, whereas significant differences were observed in EVs isolated from exposed hMSCs. These observations strongly suggested a biologic effect of BALF exposures, not a cross contamination from EVs found in the BALF samples.

The studies described herein provide evidence of the plasticity of hMSCs and their EVs and, importantly, provided a number of mechanistic hypotheses to be evaluated with respect to the differing hMSC-EV associated miRNAs. These observations provided a growing understanding of the complex interplay of inflammatory and other pathways involved in hMSC actions in the lung and provide important information towards developing more effective hMSC-based cell therapies for ARDS and other lung diseases.

### Example 9. Materials and Methods

### Human BALF Samples

Collection and processing of BALF samples from healthy volunteers (HVs) and from ARDS patients was done as previously described. In brief, HVs underwent standard fiberoptic bronchoscopy of the right middle lobe at Dartmouth University (Exclusion criteria for HVs were: history of cardiopulmonary disease, regular smoking or vaping, and use of immunomodulatory medications. BALF samples from ARDS patients without sepsis were collected prospectively as part of an unrelated clinical investigation. For the HV lavages, twenty cc sterile saline was utilized, and samples were centrifuged, and supernatants stored at −70° C. For the ARDS patient lavages, a standard 40 ml mini-BALF with sterile saline was utilized in intubated ARDS patients and BALF samples similarly centrifuged and stored.

In Vitro Exposure of hMSCs to BALF

hMSCs were obtained and cultured in MEM/EBSS medium supplemented with 1% penicillin/streptomycin and 20% fetal bovine serum in standard tissue culture incubators. The hMSCs were obtained from multiple donors and have been previously characterized according to criteria from the International Society for Cell and Gene Therapy. hMSCs were utilized at passages 3-5 and were the same as those used in a recent trial of hMSC administration in non-COVID ARDS patients and in the previous investigations of BALF effects on hMSCs actions.

miRNA and NTA Analysis

To prepare EVs to be used for miRNA and nanoparticle tracking analyses (NTA), hMSCs were seeded into 6-well plates (2×10⁵ cells/well, 2 wells/BALF sample or control) in cell culture conditions outlined above and seeded overnight. The next day, cells were washed twice with PBS and synchronized for 24 hours in serum-free medium. After synchronization, the serum-free medium was replaced with 1 ml of serum-free medium containing either individual ARDS or individual HV BALF samples at a 20% (v/v) concentration. Control hMSCs were exposed to serum-free medium only. After 5 hours incubation at 37° C. in a standard tissue culture incubator, cell culture medium was removed, cells were washed once with PBS, and 2 ml serum-free medium was added per well. After 48 hours incubation (37° C.), the conditioned medium was collected, passed through a 0.8 μm syringe filter, and processed for EV preparation, NTA, and miRNA assessments, as described below.

### Tetraspanin Expression

To assess expression of characteristic cell surface tetraspanins (CD9, CD63, and CD81) using imaging flow cytometry, 2×10⁶ cells (for each condition) were exposed as above to either ARDS or HV BALF samples or to serum-free medium (control). Given the large BALF volume (20% v/v) required for exposures and limited amounts of available BALF, pooled rather than individual HV or ARDS samples were used for these studies.

Extracellular Vesicles (EVs) Preparation and miRNA Isolation

EV isolation from the conditioned medium of BALF-exposed hMSCs or directly from the BALF samples themselves were prepared in accordance with recent recommendations from the International Society for Extracellular Vesicles. EV and EV-derived miRNAs were isolated from the conditioned medium and BALF samples using the exoRNeasy Serum/Plasma Maxi/Maxi Midi Kit (Qiagen, Germantown, MD, USA), according to manufacturer's instructions. Sample preparations were stored at −20° C. until miRNA sequencing. For NTA and imaging flow cytometry analysis, EVs were isolated using ExoQuick-TC® (cat #EXOTC50A; System Biosciences, Palo Alto, CA) according to the manufacturer's protocol to avoid interference of elution columns with NTA protocol. This is because, when preliminary NTA were performed on samples isolated with exoRNeasy kit, background contamination from the kit elution columns were noted. Because this contamination interfered with correct assessment of the particle number and size distribution using NTA, a precipitation-based isolation method (i.e., ExoQuick-TC®) not dependent on elution columns was subsequently utilized to prepare EVs for characterization by NTA and by imaging flow cytometry. This approach resulted in robust reproducible results without concern for contaminants.

### Nanoparticle Tracking Analysis (NTA)

NTA (Zeta View®, Particle Metrix Inc, Germany; 488 nm laser) was used to measure EV particle size and concentration. All samples were analyzed at 25° C. following daily instrument calibration according to the manufacturer's recommendation. Samples were diluted in ultrapure water to an appropriate concentration before analysis. Video acquisition was performed with fixed settings for all samples (scatter mode: sensitivity 85, shutter 75; fluorescence mode: sensitivity 95, shutter 32; both: minimum brightness 20, minimum size 5, and maximum size 200). Videos of all 11 positions were recorded for each sample with 5 cycles (1 cycle equals 1 s) at each position and analyzed with the Zeta View® analysis software (Version 8.03.08.02).

### Imaging Flow Cytometry

Imaging flow cytometry was performed on the AMNIS ImageStreamX® Mark II Flow Cytometer (AMNIS/Luminex, Seattle, WA, USA). In brief, antibodies were added to the samples and incubated for 1 hour at room temperature. According to the recommendations MIFlowCyt-EV guidelines, unstained EV samples (uEVs), NaCl-HEPES buffer with antibodies but without EV sample, as well as stained sample supplemented with 1% NP40 (Calbiochem, San Diego, CA, USA) were analyzed as controls. After staining without any washing, samples were diluted with PBS and analyzed using the built-in autosampler for 96-well round bottom plates. Acquisition time was selected as 5 minutes per well. Data were acquired at 60× magnification, low flow rate, and with the removed beads option deactivated. Data were analyzed as described previously with the IDEAS software (version 6.2). Fluorescent events were plotted against the side scatter (SSC). A combined mask feature was used (MC and NMC) to improve the detection of fluorescent images. Images were analyzed for coincidences (swarm detection) by using the spot counting feature. Every data point with multiple objects was excluded from the analyses. Events with low side scatter values (<500) and fluorescence intensities higher than 300 were considered as uEVs. Average concentrations were calculated according to the acquisition volume and time.

### MiRNA Sequencing and Differential Expression Analysis

EVs were isolated either from hMSCs exposed to serum-free medium (control, n=16) or to individual BALF samples obtained from ARDS patients (N=16), or HVs (N=16) or directly from pure BALF samples obtained from HVs (N=4) and ARDS patients (N=4) as described above. RNA was isolated from each EV preparation as described above and only samples that passed quality control with an A260/A280 above 1.80 were used for RNA sequencing (control=16; ARDS=12; HV=14). A total of 35 ng of eRNA (EV-derived RNA) preparation was used for miRNA sequencing performed using the HTG EdgeSeq miRNA Whole Transcriptome Assay (miRNA WTA, as per manufacturer's instructions) that measures the expression of 2,083 human miRNA transcripts using next-generation sequencing (NGS). Raw read counts for each of the 42 samples were inputted into DESeq2's to perform the differential expression analyses. Variance stabilization transformation was used to prepare data for analyses. Significance analyses of microarrays (SAM) of normalized read counts were ran using one class analysis approach to identify miRNAs over-represented in EVs derived from control hMSCs. The δ-value was set to 7 (best delta selected by software with the lowest False Discovery Rate [FDR]) using 1000 permutations, FDR cut-off was 0(%). Differential expression analysis (DEA) was performed using DESeq2 in R (R package “DESeq2”). Four comparisons were made: (1) control vs HV, (2) ARDS vs control, (3) ARDS vs HV, and (4) ARDS vs combined control+HV. Stringent selection criteria were used to reduce the FDR rate. MiRNAs deemed to be differentially expressed (DE) were chosen on the basis of a ≥2-fold change in expression (the mean expression across samples >50 read counts) and an adjusted P-value (or FDR) <0.05 after correction for multiple comparisons. MiRNAs that were consistently overrepresented were identified by the union of pairwise comparisons and visualized using Venn diagrams.

Target prediction and functional analyses were conducted using miRNet (mirnet.ca). Enriched pathways were selected on the basis of hypergeometric testing of miRNAs with FDR ≥0.05 from the Reactome. Putative relationships were obtained using DIANA-TarBase v8 (collection of experimentally supported miRNA-gene interactions).

Supervised Classification Analysis for ARDS, HV, and Control hMSC EV miRNAs

Sparse Partial Least Squares (PLS) discriminant analysis (sPLS-DA) (R package “mixOmics”) was performed to determine the most discriminative features from ~2000 miRNAs using normalized expression profiles from ARDS or HV BALF exposed or control hMSC-derived EVs, to classify samples into these categories. Briefly, the samples were split into a training set and test set using a 0.7/0.3 split. Leave-one-out cross-validation (LOOCV) was used to select the optimal parameters for the model: the number of components needed to discriminate classes (up to 4 components were considered as the number of classes (K-1) usually performs best), as well as the number of features (discriminative miRNAs) per component (5-20 features per component were tested). The combination of components and features with the lowest balanced error rate (BER) was selected for the final model (2 components with 15 and 5 features, respectively). Prediction distances used to measure classification error were computed using maximum distance. The performance of the model for the training set and the prediction of outcomes for the test set were then evaluated using the accuracy, sensitivity, and specificity for each class assignment. The area under the receiver operating characteristic (AUROC) curve for all-vs-one comparisons for PLS-DA are based on predicted maximum distances averaged over all cross-validations and are meant to complement the analysis rather than evaluate model performance.

### Statistical Analyses

### NTA and Image Flow Analyses Data

Particle sizes and counts obtained with ZetaView® were plotted in 100 μm bins on an X-axis ranging between 2.5-903 μm and the area under the curve (AUC) for each sample was calculated. Data were analyzed using one-way ANOVA, with group (control, HV, ARDS) as a factor and Bonferroni post-hoc tests were done to identify differences between the group means. The number of positively stained particles for each tetraspanin antibody (CD9, CD63, and CD81), as well as the number of double positive (CD63/CD81) were similarly analyzed within each tetraspanin type. All analyses and graphs were done using Prism (version 9.3, GraphPad software).

miRNA Data

To determine differential expression of miRNAs found in BALF-derived EVs and hMSC-derived EVs, unpaired T-tests with Welch correction (does not assume equal standard deviations) were performed for those that passed a test for normality (Kolmogorov-Smirnov). For those that were not normally distributed, two-tailed Mann-Whitney tests were performed on a miRNA-by-miRNA basis (p=0.05). Statistical analyses were performed using GraphPad Prism software. The Mann-Whitney test was used to assess differences between two groups. P-values ≤0.05 were considered as significant, except in the case of RNA sequencing data analyzed in DESeq, where a multiple hypothesis corrected FDR less than 0.05 were significant. Spearman correlations were calculated in base R, using the t distribution to calculate P-values in those cases that included ties in rank.

TABLE 3
miRNA sequences

| | | | SEQ ID |
|---|---|---|---|
| miRNA | ID/note | Sequence | NO |
| miR- | MIMAT0022272 | uucauuugccucccagccuaca | 1 |
| 664b- | | | |
| 3p | | | |
| miR- | MIMAT0022271 | ugggcuaagggagaugauugggua | 2 |
| 664b- | | | |
| 5p | | | |
| miR- | MIMAT0005589 | ucggccugaccacccaccccac | 3 |
| 1234- | | | |
| 3p | | | |
| miR- | MIMAT0004819 | uccgguucucagggcuccacc | 4 |
| 671-3p | | | |
| miR- | MIMAT0003888 | acuccagccccacagccucagc | 5 |
| 766-3p | | | |
| miR- | MIMAT0019777 | uaggggcagcagaggaccuggg | 6 |
| 4688 | | | |
| miR- | MIMAT0019018 | aaaaggcgggagaagcccca | 7 |
| 4484 | | | |
| miR- | MIMAT0027462 | cgggccggaggucaagggcgu | 8 |
| 6781- | | | |
| 5p | | | |
| miR- | MIMAT0027038 | uggggagcggcccccggguggg | 9 |
| 1343- | | | |
| 5p | | | |
| miR- | MIMAT0019069 | cccagcaggacgggagcg | 10 |
| 4530 | | | |
| miR- | MIMAT0030420 | aagggacagggagggucgugg | 11 |
| 7845- | | | |
| 5p | | | |
| miR- | MIMAT0015052 | cggggagagaacgcagugacgu | 12 |
| 3175 | | | |
| miR- | MIMAT0027512 | uguaggcaugaggcagggcccagg | 13 |
| 6806- | | | |
| 5p | | | |
| miR- | MIMAT0019979 | cauccguccgucuguccac | 14 |
| 4800- | | | |
| 3p | | | |
| miR- | MIMAT0021128 | agggaaggggacgaggguuggg | 15 |
| 5196- | | | |
| 5p | | | |
| miR- | MIMAT0004501 | agggcuuagcugcuugugagca | 16 |
| 27a- | | | |
| 5p | | | |
| miR- | MIMAT0028111 | ucggccuggggaggaggaaggg | 17 |
| 7107- | | | |
| 5p | | | |
| miR- | MIMAT0027506 | cuggggguggggggcugggcgu | 18 |
| 6803- | | | |
| 5p | | | |
| miR- | MIMAT0019229 | guggguuggggcgggcucug | 19 |
| 3940- | | | |
| 5p | | | |
| miR- | MIMAT0030996 | ggaugguugggggcggucggcgu | 20 |
| 8069 | | | |
| miR- | MIMAT0027542 | gugcgugguggcucgaggcgggg | 21 |
| 6821- | | | |
| 5p | | | |
| miR- | MIMAT0027496 | ccagggggaugggcgagcuuggg | 22 |
| 6798- | | | |
| 5p | | | |
| miR- | MIMAT0007892 | ccccagggcgacgcggcggg | 23 |
| 1915- | | | |
| 3p | | | |
| miR- | MIMAT0015010 | gagggcggguggaggagga | 24 |
| 3141 | | | |
| miR- | MIMAT0019779 | gagcaggcgaggcugggcugaa | 25 |
| 4690- | | | |
| 5p | | | |
| miR- | MIMAT0015036 | uuagggaguagaaggguggggag | 26 |
| 3162- | | | |
| 5p | | | |
| miR- | MIMAT0019033 | ugggcuggcagggcaagugcug | 27 |
| 4498 | | | |
| miR- | MIMAT0027638 | gugaguaguggcgcgcggcggc | 28 |
| 6869- | | | |
| 5p | | | |
| miR- | MIMAT0018068 | agccgcggggaucgccgaggg | 29 |
| 3648 | | | |
| miR- | MIMAT0004957 | cggcucugggucugugggga | 30 |
| 760 | | | |
| miR- | MIMAT0016901 | gggggaagaaaaggugggg | 31 |
| 4271 | | | |
| miR- | MIMAT0027686 | caggcagguguaggguggagc | 32 |
| 6893- | | | |
| 5p | | | |
| miR- | MIMAT0027355 | cucggggcaggcggcugggagcg | 33 |
| 6727- | | | |
| 5p | | | |
| miR- | MIMAT0003336 | ggcggagggaaguagguccguuggu | 34 |
| 658 | | | |
| miR- | MIMAT0015065 | agaagaaggcggucggucugcgg | 35 |
| 3185 | | | |
| miR- | MIMAT0027498 | ggggaggugugcagggcugg | 36 |
| 6799- | | | |
| 5p | | | |
| miR- | MIMAT0027474 | uggcggggguagagcuggcugc | 37 |
| 6787- | | | |
| 5p | | | |
| miR- | MIMAT0005871 | uggcagggaggcugggagggg | 38 |
| 1207- | | | |
| 5p | | | |
| miR- | MIMAT0015062 | gcuucuguaguguaguc | 39 |
| 3182 | | | |
| miR- | MIMAT0015082 | ggaggcgcaggcucggaaaggcg | 40 |
| 3197 | | | |
| miR- | MIMAT0018968 | cgucccggggcugcgcgaggca | 41 |
| 4449 | | | |
| miR- | MIMAT0027548 | guaggggagguugggccaggga | 42 |
| 6824- | | | |
| 5p | | | |
| miR- | MIMAT0027377 | cgagggguagaagagcacagggg | 43 |
| 6738- | | | |
| 5p | | | |
| miR- | MIMAT0022977 | gagggcagcguggguguggcgga | 44 |
| 4632- | | | |
| 5p | | | |
| miR- | MIMAT0019859 | gcugcgggcugcggucagggcg | 45 |
| 4734 | | | |
| miR- | MIMAT0022691 | cggguagagagggcagugggagg | 46 |
| 197- | | | |
| 5p | | | |
| miR- | MIMAT0023710 | aaggggcugggggagcaca | 47 |
| 6085 | | | |
| miR- | MIMAT0019913 | aggcaggggcuggugcugggcggg | 48 |
| 4763- | | | |
| 3p | | | |
| miR- | MIMAT0019704 | uggagagagaaaagagacagaag | 49 |
| 4644 | | | |
| miR- | MIMAT0018072 | cggcuggaggugugagga | 50 |
| 3652 | | | |
| miR- | MIMAT0027650 | ugagggacccaggacaggaga | 51 |
| 6875- | | | |
| 5p | | | |
| miR- | MIMAT0004948 | aggcagcgggguguaguggaua | 52 |
| 885-3p | | | |
| miR- | MI0000342 | ccagcucgggcagccguggccaucuuacugggcagcauuggauggagucagg | 53 |
| 200b | | ucucuaauacugccugguaaugaugacggcggagcccugcacg | |
| miR- | MI0000103 | ugcccuggcucaguuaucacagugcugaugcugucuauucuaaagguacagu | 54 |
| 101 | | acugugauaacugaaggauggca | |
| miR- | MI0000461 | caccuuguccucacgguccaguuuucccaggaaucccuuagaugcuaagaug | 55 |
| 145 | | gggauuccuggaaauacuguucuugaggucaugguu | |
| miR- | MI0000300 | ccuggccuccugcagugccacgcuccguguauuugacaagcugaguuggaca | 56 |
| 223 | | cuccaugugguagagugucaguuugucaaauaccccaagugcggcacaugcu | |
| | | uaccag | |
| miR- | MI0003134 | gauacucgaaggagagguuguccguguugucuucucuuuauuuaugaugaaac | 57 |
| 494 | | auacacgggaaaccucuuuuuuaguauc | |
| miR- | MIMAT0004891 | ugauugacauuucuguaaugg | 58 |
| 509-3p | | | |
| miR- | MIMAT0005898 | aauggauuuuuggagcagg | 59 |
| 1246 | | | |
| miR-9 | MI0000466 | cgggguugguuguuaucuuugguuaucuagcuguaugagugguguggagucuu | 60 |
| | | cauaaagcuagauaaccgaaaguaaaaauaacccca | |
| miR- | MI0000476 | cccuggcauggugugguggggcagcugguguugugaaucaggccguugccaa | 61 |
| 138 | | ucagagaacggcuacuucacaacaccagggccacaccacacuacagg | |
| miR- | MIMAT0001075 | auuccuagaaauuguucaua | 62 |
| 384 | | | |
| miR- | MIMAT0003268 | acuuacagacaagagccuugcuc | 63 |
| 600 | | | |
| miR- | MI0000477 | ccgauguguauccucagcuuugagaacugaauuccauggguugugucagugu | 64 |
| 146a | | cagaccucugaaauucaguucuucagcugggauaucucugucaucgu | |
| miR- | MI0000095 | cugggggcuccaaagugcuguucgugcagguagugugauuacccaaccuacu | 65 |
| 93 | | gcugagcuagcacuucccgagcccccgg | |
| miR- | MI0000071 | gucagaauaaugucaaagugcuuacagugcagguagugauaugugcaucuac | 66 |
| 17 | | ugcagugaaggcacuuguagcauuauggugac | |
| miR- | MI0000681 | cuguuaaugcuaaucgugauagggguuuuugccuccaacugacuccuacaua | 67 |
| 155 | | uuagcauuaacag | |
| miR- | MIMAT0000232 | acaguagucugcacauugguua | 68 |
| 199a- | | | |
| 3p | | | |
| miR- | MIMAT0000231 | cccaguguucagacuaccuguuc | 69 |
| 199a- | | | |
| 5p | | | |
| miR- | MI0000471 | cgcuggcgacgggacauuauuacuuuugguacgcgcugugacacuucaaacu | 70 |
| 126 | | cguaccgugaguaauaaugcgccguccacggca | |
| miR- | MI0000298 | ugaacauccaggucuggggcaugaaccuggcauacaauguagauuucugugu | 71 |
| 221 | | ucguuaggcaacagcuacauugucugcuggguuucaggcuaccuggaaacau | |
| | | guucuc | |
| miR- | MI0000089 | ggagaggaggcaagaugcuggcauagcuguugaacugggaaccugcuaugcc | 72 |
| 31 | | caacauauugcaucuuucc | |
| miR- | MI0000070 | gucagcagugccuuagcagcacguaaauauuggcguuaagauucuaaaauua | 73 |
| 16 | | ucuccaguauuaacugugcugcugaaguaagguugac | |
| miR- | MI0017320 | gcuggcgucggugcuggggagggcccccgggugggccucugcucuggccccuc | 74 |
| 1343 | | cuggggcccgcacucucgcucugggcccgc | |

### REFERENCES

1. Matthay M A, Zemans R L, Zimmerman G A, Arabi Y M, Beitler J R, Mercat A, et al. Acute respiratory distress syndrome. Nat Rev Dis Primers. 2019; 5 (1): 18.
2. Wilson J G, Liu K D, Zhuo H, Caballero L, McMillan M, Fang X, et al. Mesenchymal stem (stromal) cells for treatment of ARDS: a phase 1 clinical trial. Lancet Respir Med. 2015; 3 (1): 24-32.
3. Simonson O E, Mougiakakos D, Heldring N, Bassi G, Johansson H J, Dalen M, et al. In Vivo Effects of Mesenchymal Stromal Cells in Two Patients With Severe Acute Respiratory Distress Syndrome. Stem Cells Transl Med. 2015; 4 (10): 1199-213.
4. Matthay M A, Calfee C S, Zhuo H, Thompson B T, Wilson J G, Levitt J E, et al. Treatment with allogeneic mesenchymal stromal cells for moderate to severe acute respiratory distress syndrome (START study): a randomised phase 2a safety trial. Lancet Respir Med. 2018.
5. Khoury M, Cuenca J, Cruz F F, Figueroa F E, Rocco P R M, Weiss D J. Current status of cell-based therapies for respiratory virus infections: applicability to COVID-19. Eur Respir J. 2020; 55 (6).
6. Lanzoni G, Linetsky E, Correa D, Messinger Cayetano S, Alvarez R A, Kouroupis D, et al. Umbilical cord mesenchymal stem cells for COVID-19 acute respiratory distress syndrome: A double-blind, phase 1/2a, randomized controlled trial. Stem Cells Transl Med. 2021; 10 (5): 660-73.
7. Dilogo I H, Aditianingsih D, Sugiarto A, Burhan E, Damayanti T, Sitompul P A, et al. Umbilical cord mesenchymal stromal cells as critical COVID-19 adjuvant therapy: A randomized controlled trial. Stem Cells Transl Med. 2021; 10 (9): 1279-87.
8. Zhu R, Yan T, Feng Y, Liu Y, Cao H, Peng G, et al. Mesenchymal stem cell treatment improves outcome of COVID-19 patients via multiple immunomodulatory mechanisms. Cell Res. 2021; 31 (12): 1244-62.
9. Kirkham A M, Monaghan M, Bailey A J M, Shorr R, Lalu M M, Fergusson D A, et al. Mesenchymal stem/stromal cell-based therapies for COVID-19: First iteration of a living systematic review and meta-analysis: MSCs and COVID-19. Cytotherapy. 2022.
10. Lu K, Geng S T, Tang S, Yang H, Xiong W, Xu F, et al. Clinical efficacy and mechanism of mesenchymal stromal cells in treatment of COVID-19. Stem Cell Res Ther. 2022; 13 (1): 61.
11. Qu W, Wang Z, Hare J M, Bu G, Mallea J M, Pascual J M, et al. Cell-based therapy to reduce mortality from COVID-19: Systematic review and meta-analysis of human studies on acute respiratory distress syndrome. Stem Cells Transl Med. 2020; 9 (9): 1007-22.
12. Galipeau J, Sensebe L. Mesenchymal Stromal Cells: Clinical Challenges and Therapeutic Opportunities. Cell Stem Cell. 2018; 22 (6): 824-33.
13. Abreu S C, Lopes-Pacheco M, Weiss D J, Rocco P R M. Mesenchymal Stromal Cell-Derived Extracellular Vesicles in Lung Diseases: Current Status and Perspectives. Front Cell Dev Biol. 2021; 9:600711.
14. Kouroupis D, Lanzoni G, Linetsky E, Messinger Cayetano S, Wishnek Metalonis S, Lenero C, et al. Umbilical Cord-derived Mesenchymal Stem Cells modulate TNF and soluble TNF Receptor 2 (sTNFR2) in COVID-19 ARDS patients. Eur Rev Med Pharmacol Sci. 2021; 25 (12): 4435-8.
15. Chen L, Qu J, Kalyani F S, Zhang Q, Fan L, Fang Y, et al. Mesenchymal stem cell-based treatments for COVID-19: status and future perspectives for clinical applications. Cell Mol Life Sci. 2022; 79 (3): 142.
16. Walter J, Ware L B, Matthay M A. Mesenchymal stem cells: mechanisms of potential therapeutic benefit in ARDS and sepsis. Lancet Respir Med. 2014; 2 (12): 1016-26.
17. Matthay M A, Pati S, Lee J W. Concise Review: Mesenchymal Stem (Stromal) Cells: Biology and Preclinical Evidence for Therapeutic Potential for Organ Dysfunction Following Trauma or Sepsis. Stem Cells. 2017; 35 (2): 316-24.
18. Mahida R Y, Matsumoto S, Matthay M A. Extracellular Vesicles: A New Frontier for Research in Acute Respiratory Distress Syndrome. Am J Respir Cell Mol Biol. 2020; 63 (1): 15-24.
19. Fernanda Ferreira Cruz P R M R. The Potential of Factors Released from Mesenchymal Stromal Cells as Therapeutic Agents in the Lung. In: Janette K. Burgess I H H, editor. Stem Cell-Based Therapy for Lung Disease: Springer; 2019.
20. Galipeau J, Krampera M, Leblanc K, Nolta J A, Phinney D G, Shi Y, et al. Mesenchymal stromal cell variables influencing clinical potency: the impact of viability, fitness, route of administration and host predisposition. Cytotherapy. 2021; 23 (5): 368-72.
21. Kusuma G D, Carthew J, Lim R, Frith J E. Effect of the Microenvironment on Mesenchymal Stem Cell Paracrine Signaling: Opportunities to Engineer the Therapeutic Effect. Stem Cells Dev. 2017; 26 (9): 617-31.
22. Weiss D J, English K, Krasnodembskaya A, Isaza-Correa J M, Hawthorne I J, Mahon B P. The Necrobiology of Mesenchymal Stromal Cells Affects Therapeutic Efficacy. Front Immunol. 2019; 10:1228.
23. Weiss A R R, Dahlke M H. Immunomodulation by Mesenchymal Stem Cells (MSCs): Mechanisms of Action of Living, Apoptotic, and Dead MSCs. Front Immunol. 2019; 10:1191.
24. Xu A L, Rodriguez L A, 2nd, Walker K P, 3rd, Mohammadipoor A, Kamucheka R M, Cancio L C, et al. Mesenchymal Stem Cells Reconditioned in Their Own Serum Exhibit Augmented Therapeutic Properties in the Setting of Acute Respiratory Distress Syndrome. Stem Cells Transl Med. 2019; 8 (10): 1092-106.
25. Islam D, Huang Y, Fanelli V, Delsedime L, Wu S, Khang J, et al. Identification and Modulation of Microenvironment Is Crucial for Effective Mesenchymal Stromal Cell Therapy in Acute Lung Injury. Am J Respir Crit Care Med. 2019; 199 (10): 1214-24.
26. Bustos M L, Huleihel L, Meyer E M, Donnenberg A D, Donnenberg V S, Sciurba J D, et al. Activation of human mesenchymal stem cells impacts their therapeutic abilities in lung injury by increasing interleukin (IL)-10 and IL-1RN levels. Stem Cells Transl Med. 2013; 2 (11): 884-95.
27. Abreu S C, Enes S R, Dearborn J, Goodwin M, Coffey A, Borg Z D, et al. Lung Inflammatory Environments Differentially Alter Mesenchymal Stromal Cell Behavior. Am J Physiol Lung Cell Mol Physiol. 2019.
28. Abreu S C, Xisto D G, de Oliveira T B, Blanco N G, de Castro L L, Kitoko J Z, et al. Serum from Asthmatic Mice Potentiates the Therapeutic Effects of Mesenchymal Stromal Cells in Experimental Allergic Asthma. Stem Cells Transl Med. 2018.
29. Abreu S C, Hampton T H, Hoffman E, Dearborn J, Ashare A, Singh Sidhu K, et al. Differential effects of the cystic fibrosis lung inflammatory environment on mesenchymal stromal cells. Am J Physiol Lung Cell Mol Physiol. 2020; 319 (6): L908-L25.
30. Rolandsson Enes S, Hampton T H, Barua J, McKenna D H, Dos Santos C C, Amiel E, et al. Healthy versus inflamed lung environments differentially affect mesenchymal stromal cells. Eur Respir J. 2021; 58 (4).
31. Liu K D, Levitt J, Zhuo H, Kallet R H, Brady S, Steingrub J, et al. Randomized clinical trial of activated protein C for the treatment of acute lung injury. Am J Respir Crit Care Med. 2008; 178 (6): 618-23.
32. Thery C, Witwer K W, Aikawa E, Alcaraz M J, Anderson J D, Andriantsitohaina R, et al. Minimal information for studies of extracellular vesicles 2018 (MISEV2018): a position statement of the International Society for Extracellular Vesicles and update of the MISEV2014 guidelines. J Extracell Vesicles. 2018; 7 (1): 1535750.
33. Tertel T, Gorgens A, Giebel B. Analysis of individual extracellular vesicles by imaging flow cytometry. Methods Enzymol. 2020; 645:55-78.
34. Tertel T, Bremer M, Maire C, Lamszus K, Peine S, Jawad R, et al. High-Resolution Imaging Flow Cytometry Reveals Impact of Incubation Temperature on Labeling of Extracellular Vesicles with Antibodies. Cytometry A. 2020; 97 (6): 602-9.
35. Welsh J A, Van Der Pol E, Arkesteijn G J A, Bremer M, Brisson A, Coumans F, et al. MIFlowCyt-EV: a framework for standardized reporting of extracellular vesicle flow cytometry experiments. J Extracell Vesicles. 2020; 9 (1): 1713526.
36. Tusher V G, Tibshirani R, Chu G. Significance analysis of microarrays applied to the ionizing radiation response. Proc Natl Acad Sci USA. 2001; 98 (9): 5116-21.
37. Love M I, Huber W, Anders S. Moderated estimation of fold change and dispersion for RNA-seq data with DESeq2. Genome Biol. 2014; 15 (12): 550.
38. Chang L, Zhou G, Soufan O, Xia J. miRNet 2.0: network-based visual analytics for miRNA functional analysis and systems biology. Nucleic Acids Res. 2020; 48 (W1): W244-W51.
39. Karagkouni D, Paraskevopoulou M D, Chatzopoulos S, Vlachos I S, Tastsoglou S, Kanellos I, et al. DIANA-TarBase v8: a decade-long collection of experimentally supported miRNA-gene interactions. Nucleic Acids Res. 2018; 46 (D1): D239-D45.
40. Zhong X, Tang J, Li H, Shi X, Wu Y, Xia D, et al. MiR-3175 promotes epithelial-mesenchymal transition by targeting Smad7 in human conjunctiva and pterygium. FEBS Lett. 2020; 594 (7): 1207-17.
41. Silva A K A, Morille M, Piffoux M, Arumugam S, Mauduit P, Larghero J, et al. Development of extracellular vesicle-based medicinal products: A position paper of the group “Extracellular Vesicle translation to clinicaL perspectiVEs—EVOLVE France”. Adv Drug Deliv Rev. 2021; 179:114001.
42. Cruz F F, Borg Z D, Goodwin M, Sokocevic D, Wagner D E, Coffey A, et al. Systemic Administration of Human Bone Marrow-Derived Mesenchymal Stromal Cell Extracellular Vesicles Ameliorates Aspergillus Hyphal Extract-Induced Allergic Airway Inflammation in Immunocompetent Mice. Stem Cells Transl Med. 2015; 4 (11): 1302-16.
43. Younes N, Zhou L, Amatullah H, Mei S H J, Herrero R, Lorente J A, et al. Mesenchymal stromal/stem cells modulate response to experimental sepsis-induced lung injury via regulation of miR-27a-5p in recipient mice. Thorax. 2020; 75 (7): 556-67.
44. Mahida R Y, Price J, Lugg S T, Li H, Parekh D, Scott A, et al. CD14 Positive Extracellular Vesicles in Broncho-Alveolar Lavage Fluid as a New Biomarker of Acute Respiratory Distress Syndrome. Am J Physiol Lung Cell Mol Physiol. 2022.
45. de Jong T V, Moshkin Y M, Guryev V. Gene expression variability: the other dimension in transcriptome analysis. Physiol Genomics. 2019; 51 (5): 145-58.
46. Aros C J, Pantoja C J, Gomperts B N. Wnt signaling in lung development, regeneration, and disease progression. Commun Biol. 2021; 4 (1): 601.
47. Cheng L, Zhao Y, Qi D, Li W, Wang D. Wnt/beta-catenin pathway promotes acute lung injury induced by LPS through driving the Th17 response in mice. Biochem Biophys Res Commun. 2018; 495 (2): 1890-5.
48. Douglas I S, Diaz del Valle F, Winn R A, Voelkel N F. Beta-catenin in the fibroproliferative response to acute lung injury. Am J Respir Cell Mol Biol. 2006; 34 (3): 274-85.
49. Droste M, Tertel T, Jeruschke S, Dittrich R, Kontopoulou E, Walkenfort B, et al. Single extracellular vesicle analysis performed by imaging flow cytometry in contrast to NTA rigorously assesses the accuracy of urinary extracellular vesicle preparation techniques. bioRxiv. 2021:2021.04.01.437817.

### OTHER EMBODIMENTS

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

1-17. (canceled)

18. A pharmaceutical composition, comprising:
at least one mesenchymal stromal cell-derived extracellular vesicle associated miRNA (MSC-EV-miRNA);
a lipid nanoparticle (LNP) encapsulating the at least one MSC-EV-miRNA and
a pharmaceutically acceptable excipient.

19. (canceled)

20. The pharmaceutical composition of claim 18, wherein the LNP encapsulates at least four MSC-EV-miRNAs.

21. The pharmaceutical composition of claim 18, wherein the LNP encapsulates at least 14 MSC-EV-miRNAs.

22. The pharmaceutical composition of claim 18, wherein the LNP comprises a targeting moiety.

23. The pharmaceutical composition of claim 18, wherein the miRNA comprises hsa-miR-7107-5p.

24. The pharmaceutical composition of claim 18, wherein the miRNA comprises hsa-miR-6803-5p.

25. The pharmaceutical composition of claim 18, wherein the miRNA comprises hsa-miR-6798-5p.

26. The pharmaceutical composition of claim 18, wherein the miRNA comprises hsa-miR-760.

27. The pharmaceutical composition of claim 18, wherein the miRNA comprises hsa-miR-6727-5p.

28. The pharmaceutical composition of claim 18, wherein the miRNA comprises hsa-miR-4763-3p.

29. The pharmaceutical composition of claim 18, wherein the miRNA comprises hsa-miR-3652.

30. The pharmaceutical composition of claim 18, wherein the miRNA comprises hsa-miR-885-3p.

31. The pharmaceutical composition of claim 18, wherein the miRNA comprises hsa-miR-766-3p.

32. The pharmaceutical composition of claim 18, wherein the miRNA comprises hsa-miR-3175.

33. The pharmaceutical composition of claim 18, wherein the miRNA comprises hsa-miR-6893-5p.

34. The pharmaceutical composition of claim 18, wherein the miRNA comprises hsa-miR-6875-5.

35. The pharmaceutical composition of claim 18, wherein the miRNA comprises hsa-miR-6799-5p.

36. The pharmaceutical composition of claim 18, wherein the miRNA comprises miR-6787-5p, miR-200b, miR-101, miR-145, miR-223, miR-494, miR-509-3p, miR-1246, miR-9, miR-138, miR-384, miR-600, miR-146a, miR-93, miR-17, miR-155, miR-199a-3p, miR-199a-5p, miR-126, miR-221, miR-31, miR-16, and/or miR-1343.

37-38. (canceled)

39. A method of producing therapeutic mesenchymal stromal cells (MSC) for preparing the pharmaceutical composition of claim 18, the method comprising:
obtaining a first biological sample from a healthy first subject and a second biological sample from a second subject suspected of having a disease;
culturing the first biological sample in a first culture of MSCs and a second biological sample in a second culture of MSCs;
isolating hMSC-associated extracellular vesicles (hMSC-EVs) from the cell culture;
determining whether at least one EV-associated miRNA (EV-miRNA) is present in the hMSC-EVs using a next generation sequencing platform,
using the next generation sequencing platform to create a differential EV-miRNA profile, and
engineering the therapeutic MSC to overexpress one or more miRNAs identified in the EV-miRNA profile.

40. The method of claim 39, further comprising delivering the therapeutic MSC to the subject.

41. The method of claim 39, wherein the therapeutic MSC overexpresses hsa-miR-760, hsa-miR-3175, hsa-miR-885-3p, and hsa-miR-766-3p.

42-45. (canceled)


SEQUENCE LISTING (74 sequences)

SEQ ID NO: 1 - RNA, 22 nt, Homo sapiens
  source 1..22: mol_type = genomic RNA
ttcatttgcctcccagcctaca

SEQ ID NO: 2 - RNA, 24 nt, Homo sapiens
  source 1..24: mol_type = genomic RNA
tgggctaagggagatgattgggta

SEQ ID NO: 3 - RNA, 22 nt, Homo sapiens
  source 1..22: mol_type = genomic RNA
tcggcctgaccacccaccccac

SEQ ID NO: 4 - RNA, 21 nt, Homo sapiens
  source 1..21: mol_type = genomic RNA
tccggttctcagggctccacc

SEQ ID NO: 5 - RNA, 22 nt, Homo sapiens
  source 1..22: mol_type = genomic RNA
actccagccccacagcctcagc

SEQ ID NO: 6 - RNA, 22 nt, Homo sapiens
  source 1..22: mol_type = genomic RNA
taggggcagcagaggacctggg

SEQ ID NO: 7 - RNA, 20 nt, Homo sapiens
  source 1..20: mol_type = genomic RNA
aaaaggcgggagaagcccca

SEQ ID NO: 8 - RNA, 21 nt, Homo sapiens
  source 1..21: mol_type = genomic RNA
cgggccggaggtcaagggcgt

SEQ ID NO: 9 - RNA, 22 nt, Homo sapiens
  source 1..22: mol_type = genomic RNA
tggggagcggcccccgggtggg

SEQ ID NO: 10 - RNA, 18 nt, Homo sapiens
  source 1..18: mol_type = genomic RNA
cccagcaggacgggagcg

SEQ ID NO: 11 - RNA, 21 nt, Homo sapiens
  source 1..21: mol_type = genomic RNA
aagggacagggagggtcgtgg

SEQ ID NO: 12 - RNA, 22 nt, Homo sapiens
  source 1..22: mol_type = genomic RNA
cggggagagaacgcagtgacgt

SEQ ID NO: 13 - RNA, 24 nt, Homo sapiens
  source 1..24: mol_type = genomic RNA
tgtaggcatgaggcagggcccagg

SEQ ID NO: 14 - RNA, 19 nt, Homo sapiens
  source 1..19: mol_type = genomic RNA
catccgtccgtctgtccac

SEQ ID NO: 15 - RNA, 22 nt, Homo sapiens
  source 1..22: mol_type = genomic RNA
agggaaggggacgagggttggg

SEQ ID NO: 16 - RNA, 22 nt, Homo sapiens
  source 1..22: mol_type = genomic RNA
agggcttagctgcttgtgagca

SEQ ID NO: 17 - RNA, 22 nt, Homo sapiens
  source 1..22: mol_type = genomic RNA
tcggcctggggaggaggaaggg

SEQ ID NO: 18 - RNA, 22 nt, Homo sapiens
  source 1..22: mol_type = genomic RNA
ctgggggtggggggctgggcgt

SEQ ID NO: 19 - RNA, 20 nt, Homo sapiens
  source 1..20: mol_type = genomic RNA
gtgggttggggcgggctctg

SEQ ID NO: 20 - RNA, 23 nt, Homo sapiens
  source 1..23: mol_type = genomic RNA
ggatggttgggggcggtcggcgt

SEQ ID NO: 21 - RNA, 23 nt, Homo sapiens
  source 1..23: mol_type = genomic RNA
gtgcgtggtggctcgaggcgggg

SEQ ID NO: 22 - RNA, 23 nt, Homo sapiens
  source 1..23: mol_type = genomic RNA
ccagggggatgggcgagcttggg

SEQ ID NO: 23 - RNA, 20 nt, Homo sapiens
  source 1..20: mol_type = genomic RNA
ccccagggcgacgcggcggg

SEQ ID NO: 24 - RNA, 19 nt, Homo sapiens
  source 1..19: mol_type = genomic RNA
gagggcgggtggaggagga

SEQ ID NO: 25 - RNA, 22 nt, Homo sapiens
  source 1..22: mol_type = genomic RNA
gagcaggcgaggctgggctgaa

SEQ ID NO: 26 - RNA, 23 nt, Homo sapiens
  source 1..23: mol_type = genomic RNA
ttagggagtagaagggtggggag

SEQ ID NO: 27 - RNA, 22 nt, Homo sapiens
  source 1..22: mol_type = genomic RNA
tgggctggcagggcaagtgctg

SEQ ID NO: 28 - RNA, 22 nt, Homo sapiens
  source 1..22: mol_type = genomic RNA
gtgagtagtggcgcgcggcggc

SEQ ID NO: 29 - RNA, 21 nt, Homo sapiens
  source 1..21: mol_type = genomic RNA
agccgcggggatcgccgaggg

SEQ ID NO: 30 - RNA, 20 nt, Homo sapiens
  source 1..20: mol_type = genomic RNA
cggctctgggtctgtgggga

SEQ ID NO: 31 - RNA, 19 nt, Homo sapiens
  source 1..19: mol_type = genomic RNA
gggggaagaaaaggtgggg

SEQ ID NO: 32 - RNA, 21 nt, Homo sapiens
  source 1..21: mol_type = genomic RNA
caggcaggtgtagggtggagc

SEQ ID NO: 33 - RNA, 23 nt, Homo sapiens
  source 1..23: mol_type = genomic RNA
ctcggggcaggcggctgggagcg

SEQ ID NO: 34 - RNA, 25 nt, Homo sapiens
  source 1..25: mol_type = genomic RNA
ggcggagggaagtaggtccgttggt

SEQ ID NO: 35 - RNA, 23 nt, Homo sapiens
  source 1..23: mol_type = genomic RNA
agaagaaggcggtcggtctgcgg

SEQ ID NO: 36 - RNA, 20 nt, Homo sapiens
  source 1..20: mol_type = genomic RNA
ggggaggtgtgcagggctgg

SEQ ID NO: 37 - RNA, 22 nt, Homo sapiens
  source 1..22: mol_type = genomic RNA
tggcgggggtagagctggctgc

SEQ ID NO: 38 - RNA, 21 nt, Homo sapiens
  source 1..21: mol_type = genomic RNA
tggcagggaggctgggagggg

SEQ ID NO: 39 - RNA, 17 nt, Homo sapiens
  source 1..17: mol_type = genomic RNA
gcttctgtagtgtagtc

SEQ ID NO: 40 - RNA, 23 nt, Homo sapiens
  source 1..23: mol_type = genomic RNA
ggaggcgcaggctcggaaaggcg

SEQ ID NO: 41 - RNA, 22 nt, Homo sapiens
  source 1..22: mol_type = genomic RNA
cgtcccggggctgcgcgaggca

SEQ ID NO: 42 - RNA, 22 nt, Homo sapiens
  source 1..22: mol_type = genomic RNA
gtaggggaggttgggccaggga

SEQ ID NO: 43 - RNA, 23 nt, Homo sapiens
  source 1..23: mol_type = genomic RNA
cgaggggtagaagagcacagggg

SEQ ID NO: 44 - RNA, 23 nt, Homo sapiens
  source 1..23: mol_type = genomic RNA
gagggcagcgtgggtgtggcgga

SEQ ID NO: 45 - RNA, 22 nt, Homo sapiens
  source 1..22: mol_type = genomic RNA
gctgcgggctgcggtcagggcg

SEQ ID NO: 46 - RNA, 23 nt, Homo sapiens
  source 1..23: mol_type = genomic RNA
cgggtagagagggcagtgggagg

SEQ ID NO: 47 - RNA, 19 nt, Homo sapiens
  source 1..19: mol_type = genomic RNA
aaggggctgggggagcaca

SEQ ID NO: 48 - RNA, 24 nt, Homo sapiens
  source 1..24: mol_type = genomic RNA
aggcaggggctggtgctgggcggg

SEQ ID NO: 49 - RNA, 23 nt, Homo sapiens
  source 1..23: mol_type = genomic RNA
tggagagagaaaagagacagaag

SEQ ID NO: 50 - RNA, 18 nt, Homo sapiens
  source 1..18: mol_type = genomic RNA
cggctggaggtgtgagga

SEQ ID NO: 51 - RNA, 21 nt, Homo sapiens
  source 1..21: mol_type = genomic RNA
tgagggacccaggacaggaga

SEQ ID NO: 52 - RNA, 22 nt, Homo sapiens
  source 1..22: mol_type = genomic RNA
aggcagcggggtgtagtggata

SEQ ID NO: 53 - RNA, 95 nt, Homo sapiens
  source 1..95: mol_type = genomic RNA
ccagctcgggcagccgtggccatcttactgggcagcattggatggagtcaggtctctaatactgcctggtaatgatgacggcggagccctgcacg

SEQ ID NO: 54 - RNA, 75 nt, Homo sapiens
  source 1..75: mol_type = genomic RNA
tgccctggctcagttatcacagtgctgatgctgtctattctaaaggtacagtactgtgataactgaaggatggca

SEQ ID NO: 55 - RNA, 88 nt, Homo sapiens
  source 1..88: mol_type = genomic RNA
caccttgtcctcacggtccagttttcccaggaatcccttagatgctaagatggggattcctggaaatactgttcttgaggtcatggtt

SEQ ID NO: 56 - RNA, 110 nt, Homo sapiens
  source 1..110: mol_type = genomic RNA
cctggcctcctgcagtgccacgctccgtgtatttgacaagctgagttggacactccatgtggtagagtgtcagtttgtcaaataccccaagtgcggcacatgcttaccag

SEQ ID NO: 57 - RNA, 81 nt, Homo sapiens
  source 1..81: mol_type = genomic RNA
gatactcgaaggagaggttgtccgtgttgtcttctctttatttatgatgaaacatacacgggaaacctcttttttagtatc

SEQ ID NO: 58 - RNA, 21 nt, Homo sapiens
  source 1..21: mol_type = genomic RNA
tgattgacatttctgtaatgg

SEQ ID NO: 59 - RNA, 19 nt, Homo sapiens
  source 1..19: mol_type = genomic RNA
aatggatttttggagcagg

SEQ ID NO: 60 - RNA, 89 nt, Homo sapiens
  source 1..89: mol_type = genomic RNA
cggggttggttgttatctttggttatctagctgtatgagtggtgtggagtcttcataaagctagataaccgaaagtaaaaataacccca

SEQ ID NO: 61 - RNA, 99 nt, Homo sapiens
  source 1..99: mol_type = genomic RNA
ccctggcatggtgtggtggggcagctggtgttgtgaatcaggccgttgccaatcagagaacggctacttcacaacaccagggccacaccacactacagg

SEQ ID NO: 62 - RNA, 20 nt, Homo sapiens
  source 1..20: mol_type = genomic RNA
attcctagaaattgttcata

SEQ ID NO: 63 - RNA, 23 nt, Homo sapiens
  source 1..23: mol_type = genomic RNA
acttacagacaagagccttgctc

SEQ ID NO: 64 - RNA, 99 nt, Homo sapiens
  source 1..99: mol_type = genomic RNA
ccgatgtgtatcctcagctttgagaactgaattccatgggttgtgtcagtgtcagacctctgaaattcagttcttcagctgggatatctctgtcatcgt

SEQ ID NO: 65 - RNA, 80 nt, Homo sapiens
  source 1..80: mol_type = genomic RNA
ctgggggctccaaagtgctgttcgtgcaggtagtgtgattacccaacctactgctgagctagcacttcccgagcccccgg

SEQ ID NO: 66 - RNA, 84 nt, Homo sapiens
  source 1..84: mol_type = genomic RNA
gtcagaataatgtcaaagtgcttacagtgcaggtagtgatatgtgcatctactgcagtgaaggcacttgtagcattatggtgac

SEQ ID NO: 67 - RNA, 65 nt, Homo sapiens
  source 1..65: mol_type = genomic RNA
ctgttaatgctaatcgtgataggggtttttgcctccaactgactcctacatattagcattaacag

SEQ ID NO: 68 - RNA, 22 nt, Homo sapiens
  source 1..22: mol_type = genomic RNA
acagtagtctgcacattggtta

SEQ ID NO: 69 - RNA, 23 nt, Homo sapiens
  source 1..23: mol_type = genomic RNA
cccagtgttcagactacctgttc

SEQ ID NO: 70 - RNA, 85 nt, Homo sapiens
  source 1..85: mol_type = genomic RNA
cgctggcgacgggacattattacttttggtacgcgctgtgacacttcaaactcgtaccgtgagtaataatgcgccgtccacggca

SEQ ID NO: 71 - RNA, 110 nt, Homo sapiens
  source 1..110: mol_type = genomic RNA
tgaacatccaggtctggggcatgaacctggcatacaatgtagatttctgtgttcgttaggcaacagctacattgtctgctgggtttcaggctacctggaaacatgttctc

SEQ ID NO: 72 - RNA, 71 nt, Homo sapiens
  source 1..71: mol_type = genomic RNA
ggagaggaggcaagatgctggcatagctgttgaactgggaacctgctatgccaacatattgccatctttcc

SEQ ID NO: 73 - RNA, 89 nt, Homo sapiens
  source 1..89: mol_type = genomic RNA
gtcagcagtgccttagcagcacgtaaatattggcgttaagattctaaaattatctccagtattaactgtgctgctgaagtaaggttgac

SEQ ID NO: 74 - RNA, 84 nt, Homo sapiens
  source 1..84: mol_type = genomic RNA
gctggcgtcggtgctggggagcggcccccgggtgggcctctgctctggcccctcctggggcccgcactctcgctctgggcccgc